(19) United States
(12) Patent Application Publication
(10) Pub. No.: US 2019/0015351 A1
(43) Pub. Date: Jan. 17, 2019

(54) Multifunctional Nanoparticles For Prevention And Treatment Of Atherosclerosis

(71) Applicant: Drexel University, Philadelphia, PA (US)

(72) Inventors: Yinghui Zhong, Merion Station, PA (US); Jia Nong, Philadelphia, PA (US); Zhicheng Wang, Philadelphia, PA (US)

(21) Appl. No.: 16/035,054

(22) Filed: Jul. 13, 2018

Related U.S. Application Data
(60) Provisional application No. 62/532,610, filed on Jul. 14, 2017.

(51) Int. Cl. A61K 9/51; A61K 47/20; A61K 47/24; A61K 47/32; A61K 47/36; A61K 47/42; A61K 47/02; A61K 31/65; A61K 31/12; A61P 9/10

(52) U.S. Cl. CPC A61K 9/5169; A61K 9/5161; A61K 9/5138; A61K 9/5123; A61K 9/5115; A61K 47/20; A61P 9/10; A61K 47/32; A61K 47/36; A61K 47/42; A61K 47/02; A61K 31/65; A61K 31/12; A61K 47/24

(57) ABSTRACT

This disclosure relates to nanoparticles for preventing, treating and reversing atherosclerosis.

### CROSS-REFERENCE TO RELATED APPLICATIONS

This application claims the benefit of U.S. Provisional Patent Application No. 62/532,610, filed Jul. 14, 2017, which is hereby incorporated by reference.

### TECHNICAL FIELD

This disclosure relates to nanoparticles for preventing, treating and reversing atherosclerosis.

### BACKGROUND

Atherosclerosis is the leading cause of heart attack. It is a chronic inflammatory disease of the artery wall. The inflamed cells release free radicals that produce a strong local oxidative environment where low density lipoproteins (LDLs) are oxidized. The modified LDL (oxLDL) particles are endocytosed by macrophages via scavenger receptors. As a result, the macrophages develop into lipid-laden foam cells. Foam cells play a pivotal role in the occurrence and development of atherosclerosis by contributing to lipid accumulation, necrotic core expansion and further inflammatory amplification at the plaque sites. They eventually die and form part of the atherosclerotic plaque. In addition, high blood level of LDL cholesterol has been suggested to be associated with high risk of atherosclerosis, heart attack, and stroke.

Currently there is no effective therapy to treat atherosclerosis. Dextran sulfate (DS) is a biocompatible and biodegradable polysaccharide that is highly negatively charged due to its numerous sulfate groups, which can selectively bind to the positively charged apolipoprotein B molecule in LDL. Thus, it has been used in LDL apheresis, a procedure that runs a patient's blood through a machine to remove LDL cholesterol. In addition, it can bind to scavenger receptor A (SR-A). This property can potentially be used to inhibit oxLDL uptake by macrophages.

Many studies reported the development of polyelectrolyte complexes (PEC) of dextran sulfate and chitosan (CH). CH is a natural biocompatible polysaccharide with abundant amine groups that can form strong electrostatic interactions with the sulfate groups on DS. The strong electrostatic interactions between the two polymers enables the formation stable insoluble PEC of various sizes. To prolong the retention time of nanoparticles in blood circulation to increase their chance to reach the target tissue, the size of nanoparticles may be from about 10 to about 400 nm to avoid clearance of nanoparticles by liver, spleen and kidney. Studies have shown that by adding low molecular weight (LMW) chitosan into excessive high molecular weight (HMW) DS drop-by-drop, PEC nanoparticles could be formed so that DS is on the surface surrounding a chitosan core, or vice versa. However, it was found that although nanoparticles less than about 200 nm can be formed based on the formulations reported in these studies, they tend to aggregate and become larger particles after centrifugation, a necessary step to collect and purify the nanoparticles.

What are needed in the art are compounds useful for treating atherosclerosis.

### SUMMARY

In one aspect, nanoparticles having a hydrodynamic diameter of from about 10 to about 400 nm are provided. In some aspects, the nanoparticles are charged. In further aspects, the nanoparticles are negatively charged. In other aspects, the nanoparticles are positively charged. The nanoparticles comprise at least one positively charged polymer such as chitosan, gelatin type A (GA), polyethyleneimine (PEI), or chymotrypsinogen, or other positively charged polymers and at least one anionic polymer comprising sulfate groups, phosphate groups, carboxyl groups, or a combination thereof, wherein the ratio of amine groups in said positively charged polymer such as chitosan, GA, or PEI to sulfate groups, phosphate groups, carboxyl groups or a combination thereof in said at least one anionic polymer comprising negatively charged groups such as sulfate groups, phosphate groups, or carboxyl groups is less than about 1, about 0.9, about 0.8, about 0.7, about 0.6, about 0.5, about 0.4, about 0.3, about 0.2, or less than about 0.1, among others, or more than about 1, about 1.1, about 1.2, about 1.3, about 1.4, about 1.5, about 1.6, about 1.7, about 1.8, about 1.9, or more than about 2.

In another aspect, pharmaceutical compositions are provided and contain the nanoparticles discussed herein.

In a further aspect, methods for preparing the nanoparticles described herein are provided and include (i) adding said positively charged polymer such as chitosan, GA, PEI, or chymotrypsinogen to a solution comprising said anionic polymer comprising sulfate groups, phosphate groups, carboxyl groups or a combination thereof; and (ii) stirring the product of step (i); and (iii) isolating the nanoparticles.

In yet another aspect, methods for preparing nanoparticles having a hydrodynamic diameter of from about 80 to about 400 nm comprising a positively charged polymer such as chitosan, GA, PEI, or chymotrypsinogen and at least one anionic polymer, wherein the weight ratio of said positively charged polymer such as chitosan, GA, or PEI to said at least one anionic polymer is from 4:1 to about 1:8, and provide and comprise (i) mixing said chitosan, GA, or PEI with an acid to form a solution; (ii) adding the solution of step (i) to a solution comprising said at least one anionic polymer; (iii) stirring the product of step (ii); and (iv) isolating said nanoparticles.

In another aspect, methods of reducing low-density lipoprotein levels in a subject are provided and include administering the nanoparticles described herein to the subject.

In a further aspect, methods of elevating apolipoprotein-A1 (ApoA 1) production by foam cells in a subject are provided and include administering the nanoparticles described herein to the subject.

Other aspects and embodiments of the invention will be readily apparent from the following

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific compositions, methods, devices, and systems disclosed. In addition, the drawings are not necessarily drawn to scale.

FIG. 1 is a bar graph showing cell viability of three cell types related to atherosclerosis treated by DS-CH NPs without or with different metal ions fabricated by drop-by-drop method.

FIGS. 2A-2D are bar graphs for DS-CH NPs to reduce LDL level in human serum. (FIG. 2A) 0.05-1 mg/mL DS-CH NPs significantly reduced LDL cholesterol level in human serum after 24 h of treatment; (FIG. 2B) 0.5 mg/mL NP significantly reduced LDL level 5 minutes to 24 h after treatment. There was no significant difference in LDL level between 5 h and 24 h NP treatments, suggesting that 5 hours of NP treatment was sufficient to reach the maximum LDL binding capacity; (FIG. 2C) 0.5 mg/ml DS-CH-Ca NPs, DS-CH NPs, Hep-CH NPs, Hep-CH NPs, Alg-CH NPs, PP-CH NPs, DS-GA NPs and curcumin loaded NPs (CNP) containing 29 μg curcumin/mg significantly reduced LDL cholesterol level after 24 h of treatment (Hep: heparin, CS: chondrointin sulfate, Alg: alginate, PP: polyphosphate, GA: gelatin type A); and (FIG. 2D) 0.5 mg/mL negatively and positively NPs significantly reduced LDL cholesterol after 5 h of treatment. Increasing charge significantly decreased the NP's LDL binding capacity.

FIG. 3A is an oil red O staining for lipid content of mouse RAW264.7 macrophages treated with culture medium containing oxLDL and incubated with different concentrations of DS-CH nanoparticles. FIG. 3B is a bar graph showing the quantification of cholesteryl ester (CE) level in the cells. Cell culture medium containing 20 μg/mL oxLDL was incubated with different concentrations of DS-CH nanoparticles at 37° C. for 24 h. Then the medium was centrifuged to remove nanoparticles. The supernatant was used to treat mouse RAW264.7 macrophages for 24 h. Quantification of CE level in the cells shows that medium treated by 0.125-0.5 mg/mL nanoparticles for 24 h significantly reduced CE level compared to positive control, suggesting that the nanoparticles can effectively remove oxLDL from the medium. In addition, CE level in the cells cultured the medium pretreated with 0.25 or 0.5 mg/mL nanoparticles was not significantly different from negative control, suggesting that 0.25 mg/mL is sufficient to effectively remove oxLDL from the culture medium. FIG. 3C is a bar graph showing the quantification of lipid content in the cells. Quantification of lipid content in the cells confirmed that medium treated by 0.125-0.5 mg/mL nanoparticles for 24 h significantly reduced lipid content compared to positive control, suggesting that the nanoparticles can effectively remove oxLDL from the medium. In addition, lipid in the cells cultured the medium pretreated with 0.25 or 0.5 mg/mL nanoparticles was not significantly different from negative control, suggesting that 0.25 mg/mL is sufficient to effectively remove oxLDL from the culture medium.

FIG. 4A is an oil red O staining for lipid content of mouse RAW264.7 macrophages treated with culture medium containing oxLDL and incubated with 0.5 mg/mL DS-CH nanoparticles. FIG. 4B is a bar graph showing the quantification of lipid content in the cells. Cell culture medium containing 20 μg/mL oxLDL was incubated with 0.5 mg/mL DS-CH NP at 37° C. for 1, 5, and 24 h. Then the medium was centrifuged to remove nanoparticles. The supernatant was used to treat mouse RAW264.7 macrophages for 24 h. FIG. 4B also shows that medium treated by nanoparticles for 1-24 h significantly reduced lipid content compared to positive control, suggesting that the nanoparticles can effectively remove oxLDL from the medium. In addition, lipid level in the cells cultured the medium pretreated with nanoparticles for 5 or 24 h was not significantly different from negative control, suggesting that 5 h is sufficient to effectively remove oxLDL from the culture medium.

FIG. 5A is an oil red O staining for lipid content of mouse RAW264.7 macrophages treated with culture medium containing oxLDL and incubated with different concentrations of heparin (Hep)-CH nanoparticles. FIG. 5B is a bar graph showing the quantification of lipid content in the cells. Cell culture medium containing 20 μg/mL oxLDL was incubated with different concentrations of Hep-CH nanoparticles at 37° C. for 24 h. Then the medium was centrifuged to remove nanoparticles. The supernatant was used to treat Mouse RAW264.7 macrophages for 24 h. FIG. 5B confirmed that medium treated by 0.125-0.5 mg/mL nanoparticles for 24 h significantly reduced lipid content compared to positive control, suggesting that the nanoparticles can effectively remove oxLDL from the medium. In addition, lipid in the cells cultured the medium pretreated with 0.25 or 0.5 mg/mL nanoparticles was not significantly different from negative control, suggesting that 0.25 mg/mL is sufficient to effectively remove oxLDL from the culture medium.

FIG. 6 is an oil red O staining for lipid content of mouse RAW264.7 macrophages treated with culture medium containing oxLDL and incubated with 0.5 mg/mL nanoparticles. Cell culture medium containing 20 μg/mL oxLDL was incubated with 0.5 mg/mL nanoparticles at 37° C. for 24 h. Then the medium was centrifuged to remove nanoparticles. The supernatant was used to treat mouse RAW264.7 macrophages for 24 h.

FIG. 7A is an oil red O staining for lipid content of mouse RAW264.7 macrophages treated with cell culture medium containing 30 μg/mL oxLDL together with different concentrations of DS-CH nanoparticles at 37° C. for 24 h. FIG. 7B is a bar graph illustrating quantification of CE level in the cells. Quantification of CE level in the cells shows that 0.125-0.5 mg/mL nanoparticles significantly reduced CE level compared to positive control, suggesting that the nanoparticles can effectively inhibit uptake by macrophages. In addition, CE level in the cells treated by 0.25 or 0.5 mg/mL nanoparticles was not significantly different from negative control, suggesting that 0.25 mg/mL is sufficient to effectively inhibit oxLDL uptake. FIG. 7C is a bar graph illustrating quantification of lipid content in the cells. Quantification of lipid content in the cells confirmed that 0.125-0.5 mg/mL nanoparticles significantly reduced lipid content compared to positive control, suggesting that the nanoparticles can effectively inhibit oxLDL uptake by macrophages. In addition, lipid in the cells treated by 0.25 or 0.5 mg/mL nanoparticles was not significantly different from negative control, suggesting that 0.25 mg/mL is sufficient to effectively inhibit oxLDL uptake.

FIG. 8A is an oil red O staining for lipid content of mouse RAW264.7 macrophages treated with cell culture medium containing 50 μg/mL oxLDL together with nanoparticles of different charges at 37° C. for 24 h. FIG. 8B is a bar graph illustrating quantification of lipid content in cells. Quantification of lipid content in the cells shows that all the differently charged nanoparticles at 0.5 mg/mL can significantly inhibit oxLDL uptake by macrophages.

FIG. 9A is an oil red O staining for lipid content of mouse RAW264.7 macrophages treated with cell culture medium containing 50 μg/mL oxLDL together with different nanoparticles at 37° C. for 24 h. AS: alginate sulfate, PEI: polyethylenimine. FIG. 9B is a bar graph illustrating quantification of lipid content in the cells. Quantification of lipid content in the cells shows that all nanoparticles significantly reduced lipid level compared to positive control, suggesting that the nanoparticles can effectively inhibit uptake by macrophages. In addition, lipid levels in the cells treated by DS-CH NPs with or without Ca²⁺, Hep-CH NPs, or AS-CH NPs were not significantly different from negative control, suggesting that the four NPs were able to completely block oxLDL uptake. FIG. 9C is oil red O staining of mouse RAW264.7 macrophages treated with cell culture medium containing 50 μg/mL oxLDL together with 0.5 mg/mL different nanoparticles at 37° C. for 24 h. PAA: Poly(acrylic acid). FIG. 9D is a bar graph illustrating quantification of lipid content in the cells shows that all nanoparticles including DNA-CH and PAA-CH NPs significantly reduced lipid content compared to positive control.

FIG. 10 is an oil red O staining of mouse RAW264.7 macrophages treated with cell culture medium containing 30 μg/mL oxLDL and 0.5 mg/mL different nanoparticles at 37° C. for 24 h.

FIGS. 11A and 11B are oil red staining of a mouse RAW264.7 macrophages pre-treated with (A) DS-CH-Ca nanoparticles (−29.78 mV) and (B) DS-CH nanoparticles with different charges for 24 h, then the cells were washed to removing nanoparticles in the culture medium, and incubated with 25 μg/mL oxLDL for 24 h. Oil red O staining of FIG. 11A shows that DS-CH-Ca NP pre-treatment reduced oxLDL uptake by macrophages in a dose-dependent manner, although even pre-treatment with 0.5 mg/mL nanoparticles cannot completely inhibit oxLDL uptake. Oil red O staining of FIG. 11B shows that pre-treatment with 0.5 mg/mL DS-CH nanoparticles at different charges reduced oxLDL uptake by macrophages, but cannot completely inhibit oxLDL uptake. FIG. 11C is a bar graph illustrating quantification of lipid content in cells, suggesting that DS-CH nanoparticles at different charges can significantly reduce oxLDL uptake by macrophages.

FIG. 12A is an oil red O staining for lipid content for mouse RAW264.7 macrophages pre-treated with 0.5 mg/mL DS-CH nanoparticles loaded with different amounts of curcumin, then the cells were washed to remove nanoparticles in the culture medium, and incubated with 25 μg/mL oxLDL for 24 h. FIG. 12B shows that increasing curcumin loading in the NPs dose-dependently inhibited oxLDL uptake. DS-CH NPs loaded with 29 μg/mg NPs were able to completely inhibit oxLDL uptake after the NPs were removed from the culture medium.

FIG. 13A is an oil red O staining of mouse RAW264.7 macrophages treated with cell culture medium containing oxLDL together with curcumin loaded DS-CH NPs (CNPs) at 37° C. for 24 h. FIG. 13B is quantification of lipid content in the cells showing that 0.5 mg/mL CNPs can significantly inhibit oxLDL uptake by macrophages.

FIG. 14A is an oil red O staining for lipid content of mouse RAW264.7 macrophages treated with 20 μg/mL oxLDL and then incubated with nanoparticles. FIG. 14B is a bar graph illustrating quantification of CE level in the cells. Quantification of CE level in the cells shows that 0.33 or 0.5 mg/mL Ca- or Mg-DS-CH NPs significantly reduced CE level compared to positive control, suggesting that 0.33 mg/mL Ca- or Mg-NPs can effectively induce cholesterol efflux. In addition, 0.17-0.5 mg/mL DS-CH NPs without metal ions (No-ion NPs) significantly reduced CE level compared to positive control, suggesting that 0.17 mg/mL No-ion nanoparticles can effectively induce cholesterol efflux. In addition, CE level in the cells treated by 0.33 or 0.5 mg/mL of all three nanoparticles was not significantly different from negative control, suggesting that 0.33 mg/mL nanoparticles are sufficient to restore cholesterol level to normal level. FIG. 14C is a bar graph illustrating quantification of lipid content in the cells. Mouse RAW264.7 macrophages were treated with 20 μg/mL oxLDL for 24 h to induce foam cell formation. Then the foam cells were incubated with nanoparticles to allow cholesterol efflux. Quantification of lipid content in the cells shows that 0.17-0.5 mg/mL nanoparticles with or without metal ions significantly reduced lipid content compared to positive control. In addition, lipid in the cells treated by 0.33 or 0.5 mg/mL nanoparticles was not significantly different from negative control, suggesting that 0.33 mg/mL nanoparticles is sufficient to restore lipid level to normal level.

FIG. 15 is an oil red O staining for lipid content of mouse RAW264.7 macrophages treated with oxLDL and incubated with 0.5 mg/mL DS-CH nanoparticles with or without Ca²⁺. Mouse RAW264.7 macrophages were treated with 20 μg/mL oxLDL for 24 h to induce foam cell formation. Then the foam cells were incubated with 0.5 mg/mL nanoparticles for 1, 5 and 24 h to allow cholesterol efflux.

FIG. 16 is a bar graph illustrating quantification of lipid content in the cells from FIG. 15 over 1, 5, or 24 h. Quantification of lipid content in the cells shows that treating the cells with 0.5 mg/mL nanoparticles for 1, 5, or 24 h significantly reduced lipid content compared to positive control in a time-dependent manner.

FIG. 17A is an oil red O staining for lipid content of mouse RAW264.7 macrophages treated with 25 μg/mL oxLDL followed by incubation with 0.5 mg/mL nanoparticles with different charges to allow cholesterol efflux. FIG. 17B is a bar graph illustrating quantification of lipid content in the cells showing that DS-CH nanoparticles of different charges significantly reduced lipid content compared to positive control. The cholesterol efflux capacity of the NPs is inversely related to the zeta potential of the NPs. In addition, DS-CH NP containing different amounts of curcumin significantly reduced lipid content compared to positive control. The cholesterol efflux capacity of the NPs is inversely related to curcumin loading of the NPs.

FIG. 18A is an oil red O staining for lipid content. Mouse RAW264.7 macrophages were treated with 25 μg/mL oxLDL for 24 h to induce foam cell formation. Then the foam cells were incubated with 0.5 mg/mL NPs for 24 h to allow cholesterol efflux. FIG. 18B is a bar graph showing quantification of lipid content in the cells shows that treating the cells with 0.5 mg/mL DS-CH-Ca²⁺ NPs, heparin (Hep)-CH-Ca²⁺ NPs, or alginate sulfate (AS)-CH-Ca²⁺ NPs significantly reduced lipid content to negative control (NC) level. In addition, all the other NPs except DS-PEI-Ca²⁺ NPs also significantly reduced lipid level compared to positive control; however, the lipid levels were still significantly higher than NC. DS-PEI NPs were ineffective in inducing cholesterol efflux from foam cells. This result suggests that sulfate, phosphate, and carboxyl groups in the negatively charged polymers contribute to the cholesterol efflux effect of NPs.

FIG. 19A is an oil red O staining for lipid content. Mouse RAW264.7 macrophages were treated with 25 μg/mL oxLDL for 24 h to induce foam cell formation. Then the foam cells were incubated with 0.5 mg/mL NPs for 24 h to allow cholesterol efflux. FIG. 19B is a bar graph showing quantification of lipid content in the cells shows that all NPs significantly reduced lipid level compared to positive control.

FIG. 20A is an Oil red O staining for lipid content. Mouse RAW264.7 macrophages were treated with 25 μg/mL oxLDL for 24 h to induce foam cell formation. Then the foam cells were incubated with 0.5 mg/mL various NPs for 24 h to allow cholesterol efflux. FIG. 20B is a bar graph showing quantification of lipid content in the cells shows that DNA-CH and PAA-CH NPs also significantly reduced lipid level compared to positive control, confirming that NPs made from negatively charged polymers with phosphate or carboxyl groups and chitosan can induce cholesterol efflux.

FIG. 21 is an oil red O staining for lipid content of mouse RAW264.7 macrophages treated with 20 μg/mL oxLDL and incubated with 0.5 mg/mL various nanoparticles for 24 h. Mouse RAW264.7 macrophages were treated with 20 μg/mL oxLDL for 24 h to induce foam cell formation. Then the foam cells were incubated with 0.5 mg/mL nanoparticles for 24 h to allow cholesterol efflux.

FIG. 22 is a bar graph showing results from BODIPY-labeled cholesterol binding assays. DS-CH NPs with or without Ca²⁺ have high binding affinity for free cholesterol″. On the other hand, although gelatin is more hydrophilic than CH, it contains hydrophobic amino acids which may make it able to bind to cholesterol as well.

FIG. 23 is a bar graph showing the measurement of anti-inflammatory activity of NPs. lipopolysaccharide (LPS) was used to stimulate the macrophages to inflammatory phenotype, which is marked by upregulation of nitric oxide (NO), a potent inflammatory mediator and cytotoxic molecule. Griess reagent was used to measure nitrite, one of the breakdown products of NO. Macrophages were treated with LPS alone, or together with DS-CH NPs or curcumin-loaded NPs (CNP) for 2 days. FIG. 23 shows that DS-CH NP did not have any significant effect on LPS-induced NO production, whereas CNP significantly inhibited NO production. Increasing curcumin loading in CNPs dose-dependently reduced NO production, and CNP containing 29 μg curcumin/mg CNP completely inhibited NO production to untreated level.

FIG. 24A is an oil red O staining for lipid content of human monocyte THP-1 cells. Human monocyte THP-1 cells were differentiated into macrophages by PMA. The macrophages were treated with 30 μg/mL oxLDL for 24 h to induce foam cell formation. Then the foam cells were incubated with 0.5 mg/mL nanoparticles for 24 h to allow cholesterol efflux. FIG. 24B is a quantification of lipid content in the cells. 0.5 mg/mL DS-CH nanoparticles significantly reduced lipid content compared to positive control. In addition, lipid content in the cells treated by all three nanoparticles was not significantly different from negative control, suggesting that DS-CH nanoparticles were effective to induce cholesterol efflux on human macrophages as well.

FIG. 25A is an oil red O staining for lipid content of human monocyte THP-1 cells. Human monocyte THP-1 cells were differentiated into macrophages by PMA. The macrophage were treated with 30 μg/mL oxLDL for 24 h to induce foam cell formation. Then the foam cells were incubated with 0.5 mg/mL DS-CH-Ca nanoparticles for 1, 5, and 25 h to induce cholesterol efflux. FIG. 25B is a quantification of lipid content in the cells. 0.5 mg/mL DS-CH nanoparticles significantly reduced lipid content compared to positive control 5 and 25 h after NP treatment. In addition, lipid content in the cells treated with NPs for 24 h was not significantly different from negative control.

FIG. 26 are bar graphs showing quantification of lipid content in the cells. Macrophages or fibroblasts were pre-treated with 0.5 mg/mL curcumin-loaded DS-CH-Ca NPs (CNPs) for 24 h, and then incubated with spinal cord homogenate. Lipid content in the cells pre-treated with CNPs were not significantly different from negative control, suggesting that CNPs were very effective in inhibiting uptake of lipid-rich cellular and myelin debris in spinal cord homogenate by both macrophages and fibroblasts.

FIG. 27 are bar graphs showing quantification of lipid content in the cells. Macrophages or fibroblasts were incubated with spinal cord homogenate for 24 h to induce foam cell formation. Then the foam cells were treated with 0.5 mg/mL DS-CH-Ca NPs for 24 h, which significantly decreased lipid contents to negative control level, suggesting that DS-CH-Ca NPs are very effective in inducing lipid efflux from foamy macrophages and fibroblasts.

FIG. 28 is a bar graph showing quantification of lipid content in the cells. Macrophages were incubated with myelin debris for 24 h to induce foam cell formation. Then the foam cells were treated with 0.5 mg/mL DS-CH NPs for 24 h, which significantly decreased lipid content to negative control level, suggesting that DS-CH NPs are very effective in inducing lipid efflux from foamy macrophages. This result also confirms that DS-CH and DS-CH-Ca NPs has similar lipid efflux effect in the context of myelin debris.

FIG. 29A is an oil red O staining for lipid content. FIG. 29B is a bar graph showing quantification of lipid content in mouse RAW264.7 macrophages that were treated with cell culture medium containing 50 μg/mL oxLDL together with nanoparticles of different charges at 37° C. for 24 h. FIG. 29C is a bar graph showing quantification of lipid content of mouse RAW264.7 macrophages treated with 25 μg/mL oxLDL followed by incubation with 0.5 mg/mL nanoparticles to allow cholesterol efflux. These show that DS-chymo (chymotrypsinogen) NPs can also inhibit foam cell formation by inhibiting uptake of oxLDL (co-treatment experiment) and induce cholesterol efflux from foam cells (post-treatment experiment). In addition, although both DS-CH and DS-chymo NPs significantly reduced lipid content compared to positive control (PC) in both co-and post-treatment experiments, only lipid levels in the DS-CH NP group were not significantly different from negative control (NC), suggesting that DS-CH NPs are more effective than DS-chymo NPs in inhibiting oxLDL uptake by macrophages and inducing cholesterol efflux from foam cells.

### DETAILED DESCRIPTION OF ILLUSTRATIVE EMBODIMENTS

In the present disclosure the singular forms “a”, “an” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” or “substantially” it will be understood that the particular value forms another embodiment. In general, use of the term “about” or “substantially” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about” or “substantially”. In other cases, the gradations used in a series of values may be used to determine the intended range available to the term “about” or “substantially” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

DS-CH nanoparticles with or without metal ions are described that can effectively reduce LDL cholesterol level in the serum through binding to LDL cholesterol. The nanoparticles also inhibit oxLDL uptake by macrophages and/or and promote cholesterol efflux from foam cells in vitro (intracellular cholesterol was restored to normal level in 24 h, most cholesterol was removed in 5 h). To date, no nanoparticles have been used for these three applications or been shown to induce cholesterol efflux from foam cells. It was found that the DS-based nanoparticles can upregulate the expression of apoA-1 gene by 112 folds. ApoA-1 is the major component of high density lipoprotein (HDL) that participate the transport of cholesterol from foam cells to the liver. HDL is considered to be the “good cholesterol” and protect against atherosclerosis. No nanoparticles or drugs to date have such a high efficacy in increasing apoA-1 gene expression.

The inventors found that the NP formulation, such as polyanion/polycation ratio, initial polymer concentration and volume, pH, and use of ions affected NP size and could further stabilize nanoparticles. The formulations, fabrication method, and the collection method were modified to obtain nanoparticles in the desired size range after centrifugation.

Further, small volumes of high concentration of chitosan or other polycation (1.18 mg/mL in 846 μL) were added into large volume of highly diluted DS solution (0.15 mg/mL in 20 mL) dropwise or by one-shot with a constant stirring at 1,200 rpm. Such techniques allowed CH to intensively mix with DS, and the well dispersed nanoparticles also prevented further aggregation.

### I. Nanoparticles and Methods of Production

The present application provides nanoparticles having a hydrodynamic diameter that permits the nanoparticles from being retained in the subject's system. Accordingly, when administered to a subject, the nanoparticles are retained in the subject's system and are not removed by the liver. Accordingly, the nanoparticles are safe for administration to a subject. Suitably, the nanoparticles have a size which permits circulation in the blood of the subject. The nanoparticles described herein are soft, flexible, have a shape that may change, or combinations thereof. In some embodiments, the hydrodynamic diameter is unrelated to the shape of the particles. In other embodiments, the nanoparticles have a hydrodynamic diameter of from about 10 nm to about 400 nm. Other embodiments within these ranges include those ranges of from about 25 nm to about 375 nm, about 50 nm to about 350 nm, about 75 nm to about 325 nm, about 75 nm to about 250 nm, about 100 nm to about 300 nm, about 125 nm to about 275 nm, about 150 nm to about 250 nm, about 175 nm to about 225 nm, or about 150 nm to about 400 nm.

The overall charge of nanoparticles may be negative or positive. Typically, the charge of the nanoparticles is dictated by the components of the nanoparticles. In some embodiments, the nanoparticles are negatively charged. In other embodiments, the nanoparticles are positively charged.

The nanoparticles comprise one or more of a positively charged polymer that is biologically safe to the subject and is capable of forming nanoparticles and one or more of an anionic polymer. In some embodiments, the positively charged polymer is lipophilic. In other embodiments, the positively charged polymer is a chitosan, gelatin type A (GA), chymotrypsinogen, or a polyethyleneimine (PEI). In other embodiments, the positively charged polymer is a chitosan. In yet other embodiments, the chitosan is partially hydrophobic and/or contains a hydrophobic component. In further embodiments, the positively charged polymer is a GA. In still other embodiments, the positively charged polymer is a GA containing a hydrophobic component. In yet other embodiments, the positively charged polymer is a PEI. In other embodiments, the PEI is hydrophilic. In further embodiments, the positively charged polymer is chymotrypsinogen. In other embodiments, the positively charged polymer is chumotrypsinogen containing a hydrophobic component. In still further embodiments, the positively charged polymer is hydrophilic. In other embodiments, the positively charged polymer is hydrophobic. In yet other embodiments, the positively charged polymer contains a hydrophobic component. This hydrophobic component permits the nanoparticles to induce cholesterol/lipid efflux from foam cells. In further embodiments, the positively charged polymer contains at least one hydrophilic component. In still further embodiments, the positively charged polymer is a protein which contains hydrophobic amino acids.

As used herein, the term “hydrophobic component” refers to a region that cannot participate in hydrogen bonding with water molecules due to its non-polar chemical substituents. In some embodiments, acetyl substituents are hydrophobic. In other embodiments, proteins such as glycine, alanine, valine, leucine, isoleucine, proline, phenylalanine, methionine, tryptophan, or tyrosine. In further embodiments, the hydrophobic moiety is an alkyl, an aryl group such as phenyl, heteroaryl such as indolyl, heterocycle such as pyrrolidine, or nonpolar hydrogens, e.g., hydrogen atoms present on an hydrocarbon group.

The term “gelatin type A” refers to a heterogeneous mixture of water-soluble proteins of high average molecular masses, present in collagen. Proteins are extracted by boiling the relevant skin, tendons, ligaments, bones, etc. in water. Type A gelatin is derived from acid-cured tissue.

The term “chymotrypsinogen” refers to a proteolytic enzyme and a precursor (zymogen) of the digestive enzyme chymotrypsin. It is a single polypeptide chain of 245 amino acids.

The term “polyethyleneimine” refers to a polymer having—CH₂CH₂NH—repeating units. The polyethyleneimine may be linear, i.e., containing all secondary amines, or branched PEIs, i.e., containing primary, secondary and tertiary amino groups.

Proteins which contain hydrophobic amino acids are useful as the positively charged polymer. In some embodiments, the protein is a positively charged protein. Examples of positively charged proteins include, without limitation, gelatin type A, cationized gelatin, growth factors, chymotrypsinogen, or lysozyme.

In some embodiments, the nanoparticles inhibit uptake of oxLDL by macrophages. In other embodiments, the nanoparticles inhibit foam cell formation. In further embodiments, the nanoparticles induce cholesterol efflux from foam cells. In still other embodiments, the nanoparticles bind to LDL and remove it from the blood. The inventors hypothesize that the anionic groups of the anionic polymer bind to LDL or cell surface receptors responsible for cholesterol/lipid uptake. Accordingly, when the nanoparticles are cleared by the body, LDL will be removed from the blood together with the nanoparticles. The inventors also found that, when the nanoparticles contain anionic groups such as sulfate groups, the nanoparticles interact with cells differently from sulfate groups on the anionic polymer. It also is desirable for the positively charged polymer, the anionic polymer, or combination thereof to have a hydrophobic component. By doing so, the nanoparticles induce cholesterol efflux from foam cells.

In some embodiments, the anionic polymer comprises sulfate groups, phosphate groups, carboxyl groups or a combination thereof. In other embodiments, the anionic polymer comprises a high density of sulfate groups, phosphate groups, carboxyl groups or a combination thereof.

In further embodiments, the anionic polymer contains sulfate groups. In other embodiments, the anionic polymer is dextran sulfate, alginate sulfate, cellulose sulfate, chondroitin sulfate, heparin, heparin sulfate, dermatan sulfate, keratan sulfate, fucoidan, polystyrene sulfonate, or combinations thereof. In other embodiments, the anionic polymer comprises dextran sulfate or heparin or alginate sulfate or chondroitin sulfate. In further embodiments, the anionic polymer comprises dextran sulfate.

The anionic nanoparticles may also include an anionic polymer lacking sulfate groups. In some embodiments, the anionic polymer contains phosphate groups. In other embodiments, the anionic polymer includes, without limitation, polyphosphate, nucleic acids such as DNA or RNA, or combinations thereof.

In other embodiments, the anionic polymer contains carboxyl groups. In other embodiments, the anionic polymer includes, without limitation, hyaluronic acid, alginate, pectin, carboxymethyl dextran, carboxymethyl amylose, carboxymethyl cellulose, carboxymethyl beta-cyclodextrin, PAA, or combinations thereof.

In further embodiments, the anionic polymer contains sulfate groups and carboxy groups. For example, the anionic polymer containing sulfate and carboxy groups is alginate sulfate.

The at least one anionic polymer has a molecular weight having a M_{w} of greater than about 1 kDa, about 2 kDa, about 3 kDa, about 4 kDa, about 5 kDa, about 6 kDa, about 7 kDa, about 8 kDa, about 9 kDa, about 10 kDa, about 20 kDa, about 30 kDa, about 40 kDa, about 50 kDa, about 60 kDa, about 70 kDa, about 80 kDa, about 90 kDa, about 100 kDa, about 200 kDa, about 300 kDa, or greater.

The term “a chitosan” as used herein refers to a linear polysaccharide composed of randomly distributed β—(1-4)-linked D-glucosamine (deacetylated unit) and N-acetyl-D-glucosamine (acetylated unit). In some embodiments, the chitosan has amine groups. In further embodiments, the chitosan has the following structure. In other embodiments, the chitosan is chitosan or glycol chitosan. The chitosan may be produced by a number of routes known in the art including syntheses from shrimp and other crustacean shells.

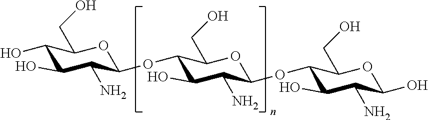

The average molecular weight of the chitosan may be determined by those skilled in the art armed with the teachings of the present application, depending on the source of the chitosan. The deacetylation (DDA, indicator of density of amino groups), molecular weight, or combinations thereof may vary. In some embodiments, the DDA and molecular weight dictate charge and size of nanoparticles (NPs). In other embodiments, the DDA of the chitosan is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%.

In some embodiments, the chitosan has an average molecular weight (M_{w}) of from about 10 to about 200 kDa. In other embodiments, the M_{w} of the chitosan is about 10, 20 30, 40, 50, 60, 65, 70, 75, 80, 85, 90, 95, 100, 110, 120, 130, 140, 150, 160, 170, 180, or 190, to about 200 kDa. As used herein, the molecular weight for the chitosan refers to the weight average molecular weight (M_{w}). The M_{w} recited herein is based on the viscosity and may also be determined by gel permeation chromatography as known in the art.

In some embodiments, the ratio of the amine groups on the chitosan to the sulfate groups or carboxyl groups on at least one anionic polymer is from less than about 1, i.e., less than about 0.9, 0.8, 0.7, 0.6, 0.5, 0.4, 0.3, 0.2, 0.1, among others. In further embodiments, the ratio of the amine groups on the chitosan to the sulfate groups or carboxyl groups on at least one anionic polymer is from more than about 1, i.e., more than about 1.1, 1.2, 1.3, 1.4, 1.5, 1.6, 1.7, 1.8, 1.9, 2, among others.

For polymers containing sulfate groups, the weight ratio of the chitosan to the anionic polymer is about 1 to 1.5: 7.5 to 8.5. In some embodiments, the weight ratio of the chitosan to the anionic polymer is about 1:3. In other embodiments, the weight ratio of the chitosan to the anionic polymer is about 1.2:3. In further embodiments, the weight ratio of chitosan to the anionic polymer is about 1:0.8.

For polymers containing phosphate groups, the weight ratio of the chitosan to the anionic polymer is greater than about 1 to 1. In some embodiments, the weight ratio of the chitosan to the anionic polymer is about 1 to 1.5: 5.5 to 6.5. In other embodiments, the weight ratio of the chitosan to the anionic polymer is about 1:4.

For polymers containing carboxyl groups, the weight ratio of the chitosan to the one anionic polymer is about 1 to 1.5: 4.5 to 5.5. In some embodiments, the weight ratio of the chitosan to the anionic polymer is about 1:3.

The nanoparticles may also be prepared by modifying existing nanoparticles to contain sulfate moieties, phosphate moieties, carboxyl moieties, or a combination thereof. For example, existing nanoparticles may be coated with sulfate groups, phosphate groups, carboxyl groups, or a combination thereof, desirably high density sulfate groups, carboxyl groups, or a combination thereof. In some embodiments, nanoparticles may be coated with sulfate groups, phosphate groups, carboxyl groups, or a combination thereof. In other embodiments, existing nanoparticles may be used, including, without limitation, gold nanoparticles, iron oxide nanoparticles, or combinations thereof such as those described in You, Carbohydrates Polymers, 2014 January 30;101:1225-33. The charge of the nanoparticles may be determined by measuring the zeta potential of the nanoparticles. One of skill in the art would readily be able to determine a zeta potential using techniques known in the art including e.g., a Zeta Sizer.

Additionally, an agent may be added to achieve the desired size of the nanoparticles. In some embodiments, metal ions may be introduced into the nanoparticles. In other embodiments, the introduction of such divalent metal ions permits loading the nanoparticles can be loaded with an active agent. In further embodiments, the metal ions are monovalent or divalent metal ions. In other embodiments, the divalent metal ion is an alkaline earth metal. In further embodiments, the alkaline earth metal is Ca²⁻, Mg²⁺,Zn²⁺, Fe²⁺, Ni²⁺, and Cu²⁺. In yet other embodiments, the monovalent ion is Na⁺. In still further embodiments, the source of the metal ion is NaCl or CaCl₂, among others. For example, a metal ion may be added to nanoparticles comprising GA.

In addition to a chitosan and anionic polymer, the nanoparticles may further comprise an active agent. The active agent may be useful in treating atherosclerosis or the like or another disease in the subject. In some embodiments, the active agent reduces local inflammation and oxidation of LDL leading to a direct effect on reducing the toxic environment present in a plaque. In other embodiments, the active agent is an antibiotic, an oxygen scavenger, anti-inflammatory, low-density lipoprotein (LDL) anti-oxidant, agent that reduces uptake of oxidized LDL, agent that increases high-density lipoprotein (HDL) release, or combinations thereof. In other embodiments, the active agent is minocycline or curcumin as said active agent.

In some embodiments, the nanoparticles comprise heparin, chondroitin sulfate, and chitosan. In other embodiments, the nanoparticles comprise dextran sulfate and chitosan. In further embodiments, the nanoparticles comprise heparin and chitosan. In yet other embodiments, the nanoparticles comprise heparin, hyaluronic acid, and chitosan. In further embodiments, the nanoparticles comprise dextran sulfate, heparin, alginate, and chondroitin sulfate. In yet other embodiments, the nanoparticles comprise dextran sulfate, heparin, chondroitin sulfate, and chitosan. In still further embodiments, the nanoparticles comprise dextran sulfate, chondroitin sulfate, and chitosan. In other embodiments, the nanoparticles comprise dextran sulfate, alginate, and chitosan. In further embodiments, the nanoparticles comprise chondroitin sulfate, hyaluronic acid, and heparin. In yet other embodiments, the nanoparticles comprise dextran sulfate, heparin, and chitosan.

The nanoparticles described herein may prepared using ordinary procedures and methods, while using the teachings described herein. The chitosan is first combined with an acid to provide a solution having a pH of about 3 to about 7. In some embodiments, the pH is about 3.5 to about 6.5. In other embodiments, the pH is about 4 to about 6. Desirably, the chitosan is dissolved in the acid. In some embodiments, the acid is acetic acid. Typically, the chitosan is added to the at least one anionic polymer. In other embodiments, the chitosan is mixed with at least one anionic polymer comprising sulfate groups and at least one anionic polymer lacking sulfate groups such as an anionic polymer containing carboxyl groups. In other embodiments, the anionic polymer solution contains about 1 about 5 mg/mL of the at least one anionic polymer. The chitosan may be poured, quickly or slowly, into the anionic polymer(s). Alternatively, the chitosan may be added dropwise to the at least one anionic polymer.

The chitosan/anionic polymer solution is then stirred for a sufficient period of time to form the nanoparticles. In some embodiments, the anionic polymer solution is stirred for at least one minute. In other embodiments, the chitosan/anionic polymer solution is stirred for at least about 5 minutes.

The nanoparticles may then be isolated using techniques known in the art. In some embodiments, the nanoparticles are isolated using filtration, centrifugation.

### II. Compositions Containing Nanoparticles

Pharmaceutical compositions useful herein, in one embodiment, contain the nanoparticles described herein in a pharmaceutically acceptable carrier or diluent with other optional suitable pharmaceutically inert or inactive ingredients. In another embodiment, the nanoparticles described herein are present in a single composition. In a further embodiment, the nanoparticles described herein are combined with one or more excipients and/or other therapeutic agents as described below.

The pharmaceutical compositions include the nanoparticles (negatively or positively) described herein described herein formulated neat or with one or more pharmaceutical carriers or excipient for administration, the proportion of which is determined by the solubility and chemical nature of the nanoparticles described herein, chosen route of administration and standard pharmacological practice. The pharmaceutical carrier may be solid or liquid. In one embodiment, pharmaceutical compositions are provided comprising the nanoparticles described herein and a pharmaceutically acceptable excipient.

The nanoparticles described herein may be administered to a subject by any desirable route, taking into consideration the specific condition for which it has been selected. The nanoparticles described herein may, therefore, be delivered by injection, i.e., transdermally, intravenously, subcutaneously, intramuscularly, intra-arterially, intraperitoneally, intracavitarily, intraparenchymally, or epiduraly, among others. In one embodiment, the nanoparticles are administered transdermally, intravenously, subcutaneously, intramuscularly, intra-arterial, intraperitoneal, intracavitary, intraparenchymally, or epiduraly.

Although the nanoparticles described herein may be administered alone, they may also be administered in the presence of one or more pharmaceutical carriers that are physiologically compatible. The carriers may be in dry or liquid form and must be pharmaceutically acceptable. Liquid pharmaceutical compositions are typically sterile solutions or suspensions.

When liquid carriers are utilized, they are desirably sterile liquids. Liquid carriers are typically utilized in preparing solutions, suspensions, emulsions, syrups and elixirs. In one embodiment, the nanoparticles described herein are dissolved a liquid carrier. In another embodiment, the nanoparticles described herein are suspended in a liquid carrier. One of skill in the art of formulations would be able to select a suitable liquid carrier, depending on the route of administration. In one embodiment, the liquid carrier includes, without limitation, water, saline, sucrose solution, organic solvents, oils, fats, or mixtures thereof. In another embodiment, the liquid carrier is water containing cellulose derivatives such as sodium carboxymethyl cellulose. In a further embodiment, the liquid carrier is water and/or dimethylsulfoxide. Examples of organic solvents include, without limitation, alcohols such as monohydric alcohols and polyhydric alcohols, e.g., glycols and their derivatives, among others. Examples of oils include, without limitation, fractionated coconut oil, arachis oil, corn oil, peanut oil, and sesame oil and oily esters such as ethyl oleate and isopropyl myristate.

Examples of excipients which may be combined with the nanoparticles (negatively or positively) described herein include, without limitation, adjuvants, antioxidants, binders, buffers, coatings, coloring agents, compression aids, diluents, disintegrants, emulsifiers, emollients, encapsulating materials, fillers, flavoring agents, glidants, granulating agents, lubricants, metal chelators, osmo-regulators, pH adjustors, preservatives, solubilizers, sorbents, stabilizers, sweeteners, surfactants, suspending agents, syrups, thickening agents, or viscosity regulators. See, the excipients described in the “Handbook of Pharmaceutical Excipients”, 5^{th} Edition, Eds.: Rowe, Sheskey, and Owen, APhA Publications (Washington, D.C.), Dec. 14, 2005, which is incorporated herein by reference.

### III. Methods of Using the Nanoparticles

The terms “patient” or “subject” as used herein refer to a mammalian animal. In one embodiment, the patient or subject is a human. In another embodiment, the patient or subject is a veterinary or farm animal, a domestic animal or pet, or animal normally used for clinical research. In still a further embodiment, the subject or patient has atherosclerosis. The subject or patient has either been recognized as having or at risk of having atherosclerosis.

As noted above, the nanoparticles bind to cholesterol including LDL cholesterol, inhibit oxLDL uptake, induce cholesterol efflux, or a combination thereof. In some embodiments, the major functional component in the nanoparticles is the negatively charged groups that permit the nanoparticles to bind to LDL or cell surface receptors; thus, the nanoparticles may be used to LDL binding and inhibiting oxLDL uptake. In other embodiments, the negatively charged groups and a hydrophobic component are the major functional components that permit the nanoparticles to induce efflux process, serve as a carrier to accept effluxed cholesterol, or a combination thereof. In fact, the inventors discovered that effluxed cholesterol binds to the nanoparticles in cell culture medium.

In further embodiments, the nanoparticles influence the levels of apolipoprotein A-1 in the subject. Examples of nanoparticles discussed herein which influence levels of apo A-1 include, without limitation, DS-CH-Ca, DS-CH, Hep-CH, Alg-CH, PP-CH, and DS-GA. In some embodiments, ApoA-1 production by foam cells in a subject. In other embodiments, the levels of ApoA-1 are increased locally at the plaque where there are foam cells.

As used herein, “treatment” encompasses treatment of a subject clinically diagnosed as having a disease or medical condition. In one embodiment, the subject is treated and the disease or medical condition is eradicated, i.e., the subject is cured. As used herein, “prevention” encompasses prevention of symptoms in a subject who has been identified as at risk for the condition, but has not yet been diagnosed with the same and/or who has not yet presented any symptoms thereof.

As described herein, a therapeutically or prophylactically effective amount of the nanoparticles is that amount which can lower blood LDL level and lessen degree of atherosclerosis. In one embodiment, the amount (i.e., per unit) of the nanoparticles described herein is that which does not exceed normal organ dose limits. In one embodiment, the dose of the nanoparticles described herein is dependent on the severity of the disease or condition being treated. In another embodiment, the dose of the nanoparticles described herein is the maximum dose tolerated by the subject. However, the effective amount to be used is subjectively determined by the attending physician and variables such as the size, age and response pattern of the subject.

These effective amounts may be provided on regular schedule, i.e., daily, weekly, monthly, or yearly basis or on an irregular schedule with varying administration days, weeks, months, etc. Alternatively, the effective amount to be administered may vary. In one embodiment, the effective amount for the first dose is higher than the effective amount for one or more of the subsequent doses. In another embodiment, the effective amount for the first dose is lower than the effective amount for one or more of the subsequent doses.

In one embodiment, methods of preventing atherosclerosis are provided and include administering the nanoparticles described herein to the subject.

In a further embodiment, methods of reducing low density lipoprotein levels in a subject are provided and include administering the nanoparticles described herein to the subject.

In yet another embodiment, methods of elevating apolipoprotein-A1 levels in a subject are provided and include administering the nanoparticles described herein to the subject.

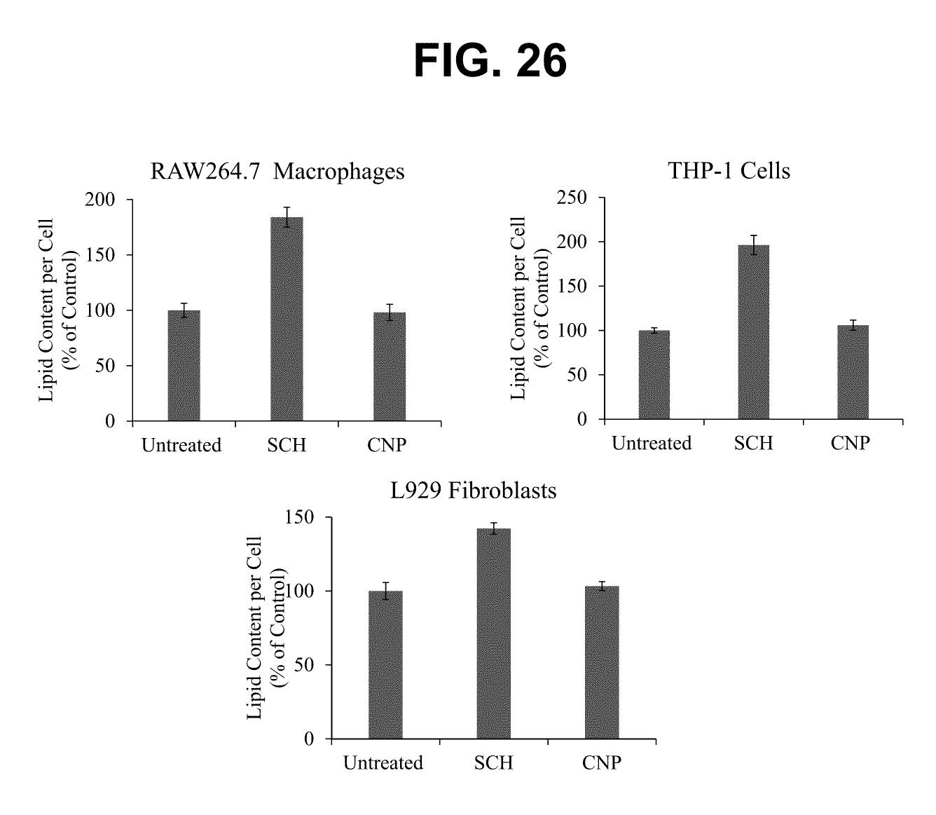

In other embodiments, methods of treating spinal cord injury in a subject are provided and include administering the nanoparticles described herein to said subject. After traumatic spinal cord injury (SCI), the injury site is filled with lipid-rich cellular and myelin debris. See, Zhu Y, et al., “Macrophage Transcriptional Profile Identifies Lipid Catabolic Pathways That Can Be Therapeutically Targeted after Spinal Cord Injury,” J. Neurosci., March 2017, 37(9):2362-2376. Macrophages are the predominant phagocyte that are responsible for debris-clearance and become lipid-laden foam cells. Recent studies show that the foam cells are unable to sufficiently clear myelin debris and become pro-inflammatory and neurotoxic. See, Wang X, et al. “Macrophages in spinal cord injury: phenotypic and functional change from exposure to myelin debris,” 2015, Glia 63:635-51 and Kroner A, et al., “TNF and increased intracellular iron alter macrophage polarization to a detrimental M1 phenotype in the injured spinal cord,” 2014, Neuron., 83(5):1098-1116. In addition, fibroblasts also infiltrate the injured spinal cord and become lipid-laden foam cells that are pro-inflammatory. Thus, the inventors found that the DS-CH NPs can effectively induce cholesterol efflux from spinal cord debris-induced foam cells, and DS-CH NPs loaded with curcumin can effectively inhibit debris uptake by macrophages and fibroblasts to prevent foam cell formation. See, FIG. 26 which shows that curcumin-loaded DS-CH NPs inhibit uptake of spinal cord debris by macrophages and fibroblasts, and the DS-CH NPs are effective in inducing lipid efflux by foamy macrophages and fibroblasts. In view of this data, it is expected that all of the NPs described herein that can induce cholesterol/lipid efflux from oxLDL-induced foam cells should be effective on spinal cord debris-induced foam cells as well. In addition, since curcumin-loaded NPs are more effective than NPs in inhibiting lipid uptake, it is expected that all of the NPs described herein loaded with curcumin should be effective in inhibiting lipid uptake regardless whether the lipid is from oxLDL or spinal cord debris.

### IV. Kits Containing the Nanoparticles

Also provided herein are kits or packages of pharmaceutical formulations containing the nanoparticles described herein or compositions containing the same. The kits may be organized to indicate a single formulation or combination of formulations to be taken at each desired time. The composition may also be sub-divided to contain appropriate quantities of the nanoparticles described herein. For example, the unit dosage can be packaged compositions, e.g., packeted powders, vials, ampoules, prefilled syringes or sachets containing liquids.

Suitably, the kit contains packaging or a container with the nanoparticles described herein formulated for the desired delivery route. Suitably, the kit contains instructions on dosing and an insert regarding the nanoparticles (negatively or positively) described herein. Optionally, the kit may further contain instructions for monitoring circulating levels of product and materials for performing such assays including, e.g., reagents, well plates, containers, markers or labels, and the like. Such kits are readily packaged in a manner suitable for treatment of a desired indication. For example, the kit may also contain instructions for use of the delivery device. Other suitable components to include in such kits will be readily apparent to one of skill in the art, taking into consideration the desired indication and the delivery route. The doses are repeated daily, weekly, or monthly, for a predetermined length of time or as prescribed.

The nanoparticles described herein or compositions containing same can be a single dose or for continuous or periodic discontinuous administration. For continuous administration, a package or kit can include the nanoparticles (negatively or positively) described herein in each dosage unit (e.g., solution, lotion, tablet, pill, or other unit described above or utilized in drug delivery). When the nanoparticles described herein are to be delivered with periodic discontinuation, a package or kit can include placebos during periods when the nanoparticles described herein are not delivered. When varying concentrations of a composition, of the components of the composition, or of relative ratios of the nanoparticles described herein or other agents within a composition over time is desired, a package or kit may contain a sequence of dosage units, so varying.

A number of packages or kits are known in the art for the use in dispensing pharmaceutical agents. In one embodiment, the package has indicators for each period. In another embodiment, the package is a labeled blister package, dial dispenser package, or bottle.

The packaging means of a kit may itself be geared for administration, such as an inhalant, syringe, pipette, eye dropper, or other such like apparatus, from which the formulation may be applied to an infected area of the body, injected into a subject, or even applied to and mixed with the other components of the kit.

The nanoparticles described herein or compositions of these kits also may be provided in dried or lyophilized forms. When reagents or components are provided as a dried form, reconstitution generally is by the addition of a suitable solvent. It is envisioned that the solvent also may be provided in another packaging means.

The kits may include a means for containing the vials in close confinement for commercial sale such as, e.g., injection or blow-molded plastic containers into which the desired vials are retained.

Irrespective of the number or type of packages, the kits also may include, or be packaged with a separate instrument for assisting with the injection/administration or placement of the ultimate complex composition within the body of an animal. Such an instrument may be an inhalant, syringe, pipette, forceps, measuring spoon, eye dropper or any such medically approved delivery means. Other instrumentation includes devices that permit the reading or monitoring of reactions in vitro.

In one embodiment, a pharmaceutical kit is provided and contains the nanoparticles described herein. The nanoparticles described herein may be in the presence or absence of one or more of the carriers or excipients described above. The kit may optionally contain other active agents and/or instructions for administering the active agent and the nanoparticles described herein to a subject.

The following Examples are provided to illustrate some of the concepts described within this disclosure. While each Example is considered to provide specific individual embodiments of composition, methods of preparation and use, none of the Examples should be considered to limit the more general embodiments described herein.

In the following examples, efforts have been made to ensure accuracy with respect to numbers used (e.g. amounts, temperature, etc.) but some experimental error and deviation should be accounted for. Unless indicated otherwise, temperature is in degrees C, pressure is at or near atmospheric.

### EXAMPLES

All the chemicals were obtained from Sigma-Aldrich and used without further purification.

### Example 1

### Nanoparticle Preparation

The following solutions were prepared for nanoparticle formation. Low molecular weight CH (50,000-190,000 daltons) was dissolved in 0.6% (v/v) acetic acid, and all other solutions were dissolved in deionized (DI) water, unless otherwise stated. All solutions were filtered through a 0.22 μm syringe filter (Millipore) before use. Solution 1 was DS (or other polyanions) solution in 20 mL of DI water, unless otherwise stated; and the solution 2 was composed of chitosan solution (or other polycations), DI water, and calcium chloride or magnesium chloride solutions for the calcium nanoparticles (Ca nanoparticles) and the magnesium nanoparticles (Mg nanoparticles) respectively, or same volume of DI water for the none ion contained nanoparticles (Non-ion nanoparticles). There will be changes in volume or concentration if necessary. Tables 1-3 list the detailed compositions of solution 1 and solution 2.

TABLE 1
Formulation of pure particles

| Nanoparticle Composition | Components in Solution 1 | Components in Solution 2 |
|---|---|---|
| DS-CH NP | DS | 250 μL CH (4 mg/mL) |
| (DS:CH weight ratio 3:1) | (0.15 mg/mL or 0.2 mg/mL in | 500 μL DI water |
| | 15 mL of DI water for larger | 96 μL metal ion solution (10 mg/mL) |
| | size)² | or DI water |
| DS-CH NP | DS | 250 μL CH (4 mg/mL) |
| (DS-CH weight ratio 1.3:1) | (0.065 mg/mL) | 500 μL DI water |
| | | 96 μL metal ion solution (10 mg/mL) |
| | | or DI water |
| DS-CH NP | DS | 250 μL CH (4 mg/mL) |
| (DS-CH weight ratio 0.8:1) | (0.04 mg/mL) | 500 μL DI water |
| | | 96 μL metal ion solution (10 mg/mL) |
| | | or DI water |
| Heparin-CH (Hep-CH) NP | Hep (0.15 mg/mL) | 250 μL CH (4 mg/mL) |
| | | 500 μL DI water |
| | | 72 μL metal ion solution (10 mg/mL) |
| | | or DI water |
| Chondroitin Sulfate-CH | CS | 250 μL CH (4 mg/mL) |
| (CS-CH) NP | (0.15 mg/mL or | 500 μL DI water |
| | 0.3 mg/mL in 10 mL DI | 48 μL metal ion solution (10 mg/mL) |
| | water for larger size) | or DI water |
| Alginate-CH (Alg-CH) NP¹ | Alg (0.15 mg/mL) | 250 μL CH (4 mg/mL) |
| | | 500 μL DI water |
| Hyaluronic Acid-CH | HA | 250 μL CH (4 mg/mL) |
| (HA-CH) NP¹ | (0.15 mg/mL) | 500 μL DI water |
| | | 96 μL metal ion solution (10 mg/mL) |
| | | or DI water |
| Polystyrene Sulfonate-CH | PSS | 250 μL CH (4 mg/mL) |
| (PSS-CH) NP | (0.15 mg/mL) | 500 μL DI water |
| | | 96 μL metal ion solution (10 mg/mL) |
| | | or DI water |
| Alginate Sulfate-CH | AS | 250 μL CH (4 mg/mL) |
| (AS-CH) NP | (0.15 mg/mL) | 500 μL DI water |
| | | 96 μL metal ion solution (10 mg/mL) |
| | | or DI water |
| Polyphosphate-CH | PP (0.2 mg/mL) | 250 μL CH (4 mg/mL) |
| (short chain, PP-CH) NP | | 500 μL DI water |
| | | 96 μL metal ion solution (10 mg/mL) |
| | | or DI water |
| Poly(acrylic acid)-CH | PAA (0.15 mg/mL, pH = 7) | 250 μL CH (4 mg/mL) |
| (PAA-CH) NP | | 500 μL DI water |
| | | 96 μL metal ion solution (10 mg/mL) |
| | | or DI water |
| DS-Gelatin Type A | DS | 300 μL GA (5 mg/mL in 0.6% acetic |
| (GA-CH) NP | (0.15 mg/mL or 0.2 mg/mL | acid) |
| | in 15 mL DI water for larger | 500 μL DI water |
| | size) | 75 μL metal ion solution (10 mg/mL) |
| | | or DI water |
| DS-Polyethyleneimine | DS | 37.5 μL PEI (1% PEI in DI water, pH = |
| (low molecular weight, DS-PEI) | (1.3 mg/mL in 2.25 mL DI | 7) |
| NP | water or 2 mg/mL in 1.5 mL | 500 μL DI water |
| | DI water for larger size) | 96 μL metal ion solution (10 mg/mL) |
| | | or DI water |
| DS-Chymotrypsinogen | DS (0.15 mg/mL) | 250 μL Chymo (4 mg/mL in DI |
| (DS-Chymo) NP³ | | water) |
| | | 500 μL DI water |

¹Glycol chitosan can be used to replace regular chitosan
²Concentration of DS in Solution 1 is 0.15 mg/mL as the formulation of DS:CH weight ratio as 3:1, and such formulation is used in most of the experiments unless otherwise stated.
³DS-Chymo NPs are collected by centrifugation at 14,000 rpm for 5 min

TABLE 2
Formulation of drug-loaded nanoparticles

| Nanoparticle | | |
|---|---|---|
| Composition | Solution 1 | Solution 2 |
| Curcumin-loaded NP | polyanion (0.15 mg/mL) | 250 μL CH (4 mg/mL) |
| | | 20, 30, or 60 μL curcumin solution (2 mg/mL |
| | | in EtOH) for nanoparticles with different |
| | | curcumin loading (7, 11, or 29 μg/mg NPs)⁴ |
| | | 96 μL metal ion solution/DI water |
| Minocycline- | polyanion (0.15 mg/mL) | 300 μL CH (4 mg/ml) |
| loaded NP | | 150 μL minocycline solution (1 mg/mL in |
| | | water) |
| | | 300 μL metal ion solution/DI water |

⁴Curcumin solution used in curcumin-loaded NP is 60 μL as 120 μg of curcumin, and such formulation is used in most of the experiments unless otherwise stated. Curcumin used in the following study was Sigma 8203540010, unless otherwise specified.

TABLE 3
Formulation of combined nanoparticles

| Nanoparticle | | |
|---|---|---|
| Composition | Solution 1 | Solution 2 |
| DS-CS-CH | 750 μL DS (4 mg/mL) | 600 μL CH (4 mg/mL) |
| | 750 μL CS (4 mg/mL) | 192 μL metal ion solution/DI |
| | 17.78 mL DI water | water |
| | | 1000 μL DI water |
| DS-Hep-CH | 750 μL DS (4 mg/mL) | 600 μL CH (4 mg/mL) |
| | 750 μL CS (4 mg/mL) | 192 μL metal ion solution/DI |
| | 17.78 mL DI water | water |
| | | 1000 μL DI water |
| DS-Alg-CH | 750 μL DS (4 mg/mL) | 600 μL CH (4 mg/mL) |
| | 750 μL Alg (1 mg/mL) | 192 μL metal ion solution/DI |
| | 17.78 mL DI water | water |
| | | 1000 μL DI water |
| DS-HA-CH⁵ | 1500 μL DS (4 mg/mL) | 400 μL glycol chitosan |
| | 1500 μL HA (1 mg/mL) | (4 mg/mL) |
| | 17 mL DI water | 384 metal ion solution/DI |
| | | water |
| | | 1000 ul DI water |
| Hep-HA-CH⁵ | 1500 μL Hep (4 mg/mL) | 500 μL glycol chitosan |
| | 1500 μL HA (1 mg/mL) | (4 mg/mL) |
| | 17 mL DI water | 192 metal ion solution/DI |
| | | water |
| | | 1000 μL DI water |

⁵Glycol chitosan was introduced in the system to replace chitosan

Two methods were used to prepare the NP: solution 2 was added either drop-wise or one-shot into 20 mL of the solution 1 with a constant magnetic stirring of 1200 rpm. The first method is referred as drop-by-drop method and the second as one-shot method. The result NP solution was continued to be stirred for 5 more min for optimal NP formation. Then it was centrifuged at 6,000 rpm for 5 min, unless otherwise stated. The supernatant was discarded. The precipitated nanoparticles were re-suspended in DI water characterization or in cell culture medium for cell treatment.

### Example 2

### Nanoparticle Characterization

Nanoparticles were characterized by hydrodynamic diameter (Dₕ), polydispersity index (PDI) and zeta potential, using a Nano ZS90 zeta-sizer (Malvern). Tables 4 and 5 summarized the characterization of the nanoparticles by two preparation methods. Drop-by-drop method is adding one solution to another oppositely charged solution drop by drop, while homogenized method is to add the solution by another one by one fast shot. If not specified, all the experiments below used drop-by-drop nanoparticles. Table 6 summarized the characterization of the nanoparticles using different weight ratio of DS and CH.

These results show that by using the same volume, homogenized method result in smaller size than drop-by-drop method, and that by altering the DS:CH weight ratio, NP size and zeta potential can be adjusted. In addition, it was found that adding Ca²⁺ or Mg²⁺ into the nanoparticles can reduce NP size. See, Tables 4-6.

TABLE 1

| | Drop-by-drop | | | | | | | | | |
|---|---|---|---|---|---|---|---|---|---|---|
| | DS-CH- | DS-CH- | DS-CH- | Hep-CH- | AlgS-CH- | CS-CH- | PP-CH- | DS-PEI- | DS-GA- | Alg-CH- |
| | Ca | Mg | Non-ion | Ca | Ca | Ca | Ca | Ca | Ca | No ion |
| Dh (nm) | 155.5 | 160.8 | 198.2 | 189.8 | 232.6 | 158.1 | 227.1 | 205.4 | 165.3 | 317.9 |
| PDI | 0.19 | 0.18 | 0.21 | 0.15 | 0.227 | 0.031 | 0.266 | 0.133 | 0.089 | 0.295 |
| Zeta potential (mV) | −20.8 | −23.1 | −34 | −21.5 | −21.7 | −20 | −33.4 | −22.9 | −27.4 | −40.5 |

TABLE 2

| | Homogenized | | | | | |
|---|---|---|---|---|---|---|
| | DS-CH- | DS-CH- | DS-CH- | Hep-CH- | CS-CH- | PSS-CH- |
| | Ca | Mg | Non-ion | Ca | Ca | Ca |
| Dh | 138.3 | 156.5 | 186.1 | 161.1 | 154.2 | 152.4 |
| PDI | 0.15 | 0.16 | 0.2 | 0.15 | 0.12 | 0.15 |

TABLE 3

| Weight Ratio DS:CH | 6.2:1 | 3:1 | 2.5:1 | 2:1 | 1.5:1 | 1.3:1 | 1:1 | 0.9:1 | 0.8:1 |
|---|---|---|---|---|---|---|---|---|---|
| Dh (nm) | 296.53 | 193.27 | 176.33 | 168.70 | 147.57 | 147.3 | 178.87 | 2848.33 | 233.27 |
| Zeta Potential (mV) | −34.58 | −29.78 | −29.10 | −26.90 | −25.40 | −24.67 | −23.48 | −11.25 | 21.88 |

### Example 3

### Cytocompatibility Assay

Mouse RAW264.7 macrophages (ATCC® TIB-71™) and bovine aortic endothelial cells (BAECs) were grown in Dulbecco's modified Eagle's medium (DMEM, with 4.5 g/L glucose and L-glutamine) supplemented with 10% fetal bovine serum (FBS) and 1% Penicillin Streptomycin (P/S). Porcine smooth muscle cells (SMCs) were cultured in DMEM (with 1 g/L glucose, L-glutamine and sodium pyruvate) supplemented with 10% FBS and 1% P/S. The cell lines described herein may be obtained from publicly available sources including, without limitation, American Type Culture collection, Thermofisher, among others.

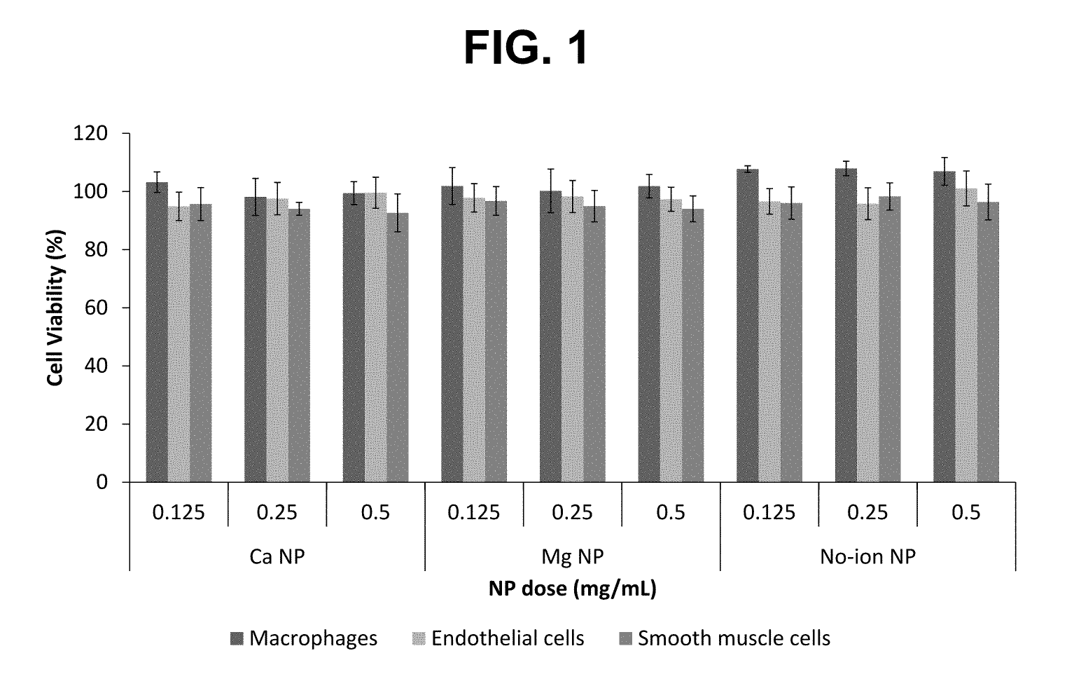

The cytocompatibility of the nanoparticles was carried out by comparing the viability of the cells treated with various concentrations of the nanoparticles. Macrophages, BAECs and SMCs were plated in 96-well plates that were pre-coated with 15 μg/mL poly-d-lysine (PDL) and allowed to attach for overnight. Then the cells were treated with nanoparticles for 24 h. Cell counting kit-8 (Dojindo) was used to determine the cell viability. See, FIG. 1. No cytotoxicity has been revealed in this study.

### Example 4

### LDL Cholesterol Binding Assay

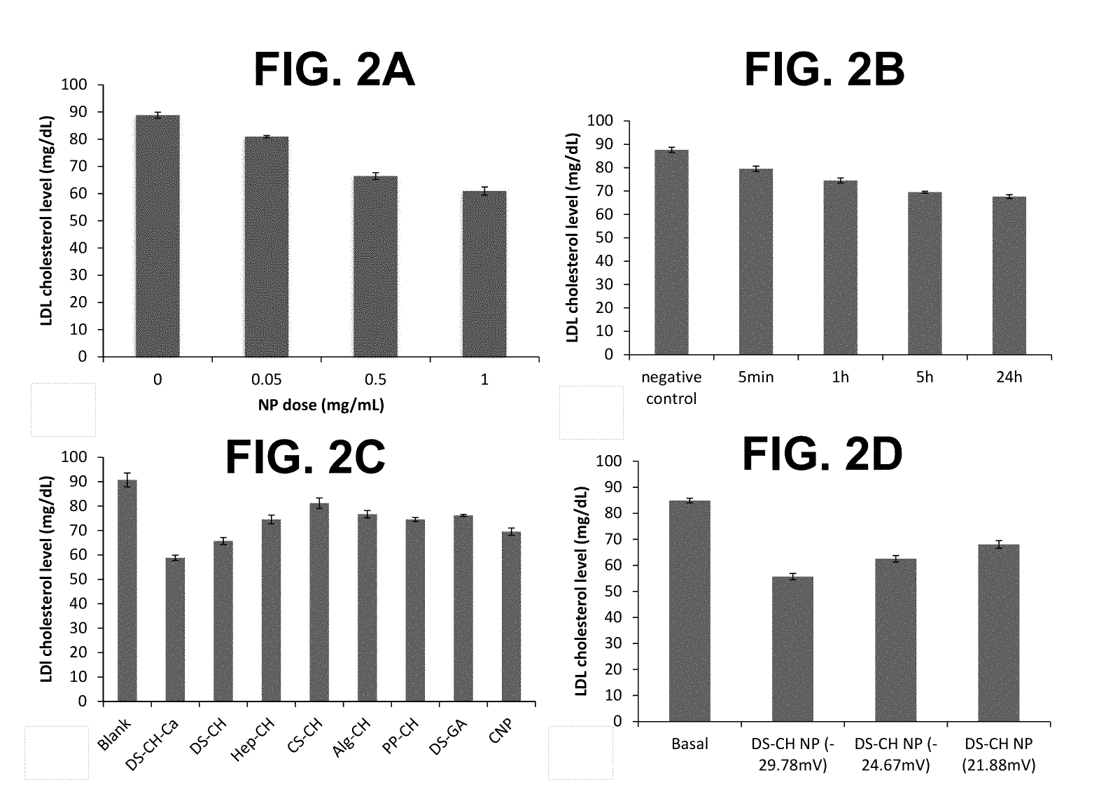

The ability of the nanoparticles to bind to LDL cholesterol was assessed by mixing the nanoparticles with human serum (Atlanta Biologicals) and incubated at 37° C. At different time points, the mixture was centrifuged at 10,000 rpm for 10 min, and the unbound LDL cholesterol level in the supernatant was measured using a LDL cholesterol assay kit (BIOO SCIENTIFIC, Austin, Tex.). See, FIG. 2. Nanoparticles could significantly reduced LDL cholesterol level in human serum.

### Example 5

### OxLDL Binding Assay (Medium-Treatment)

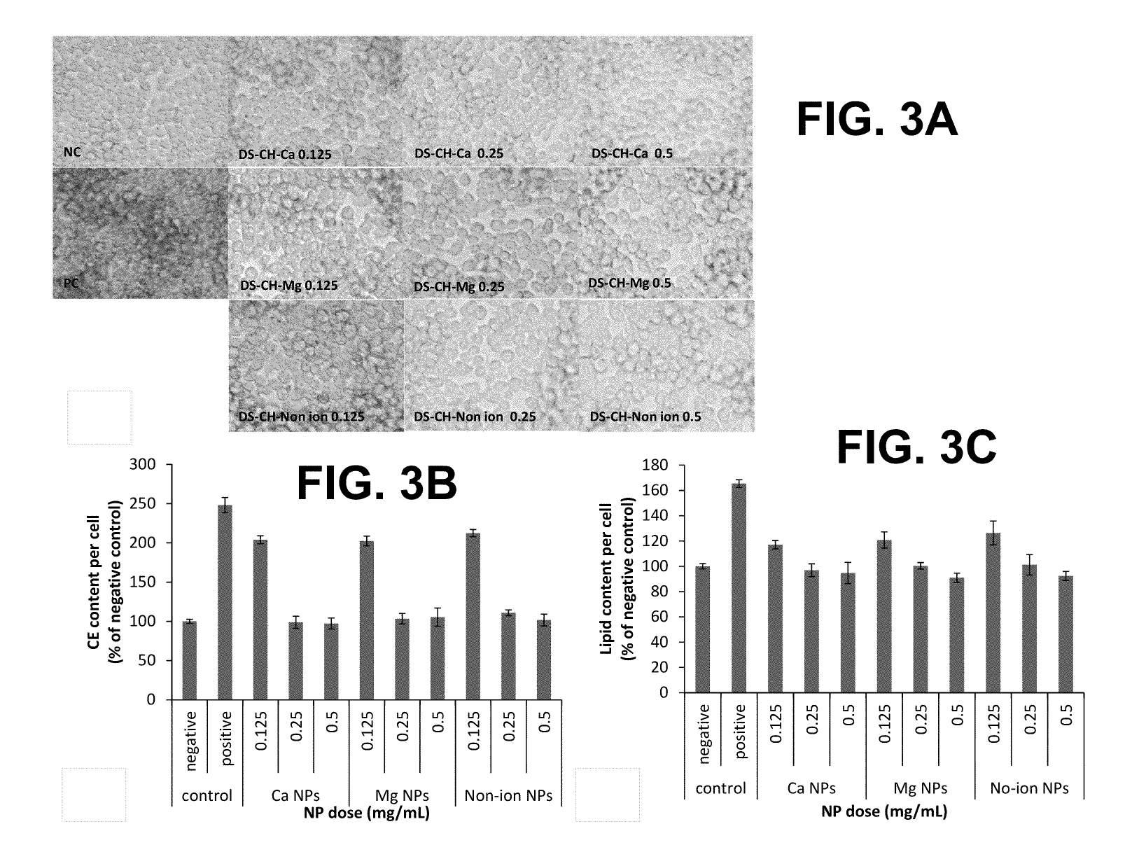

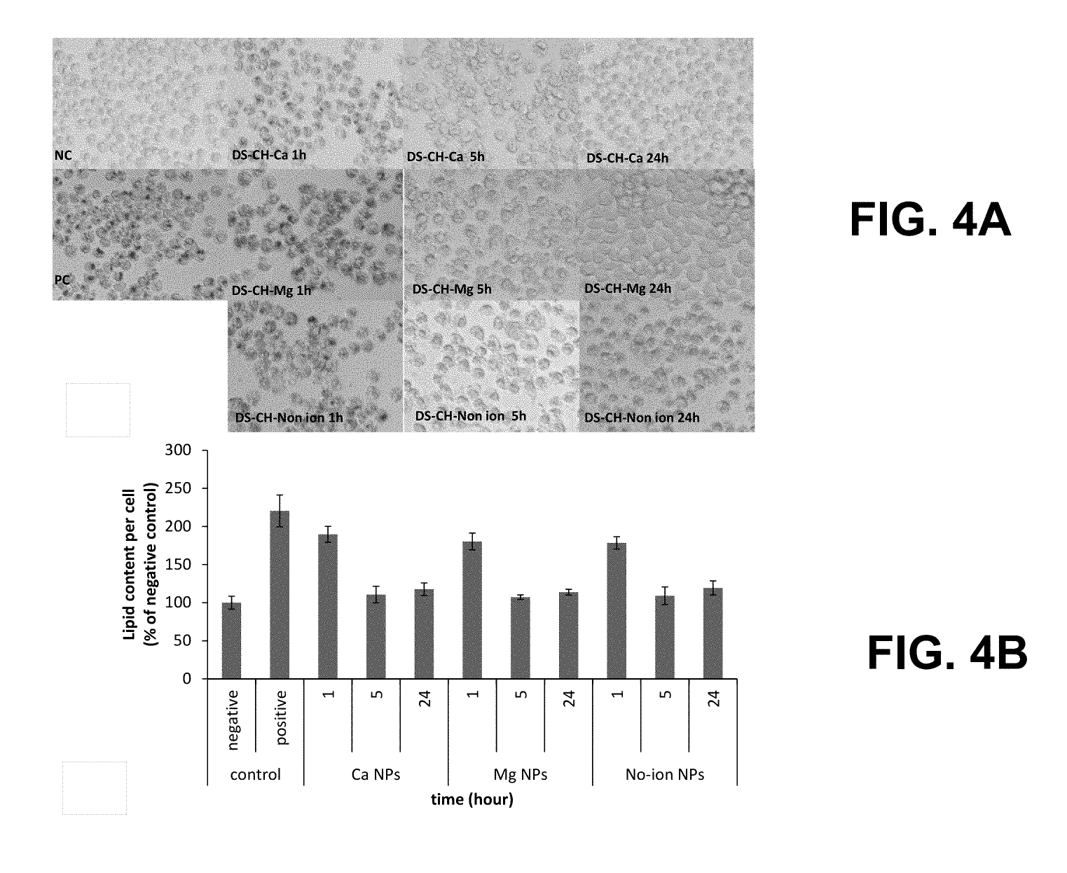

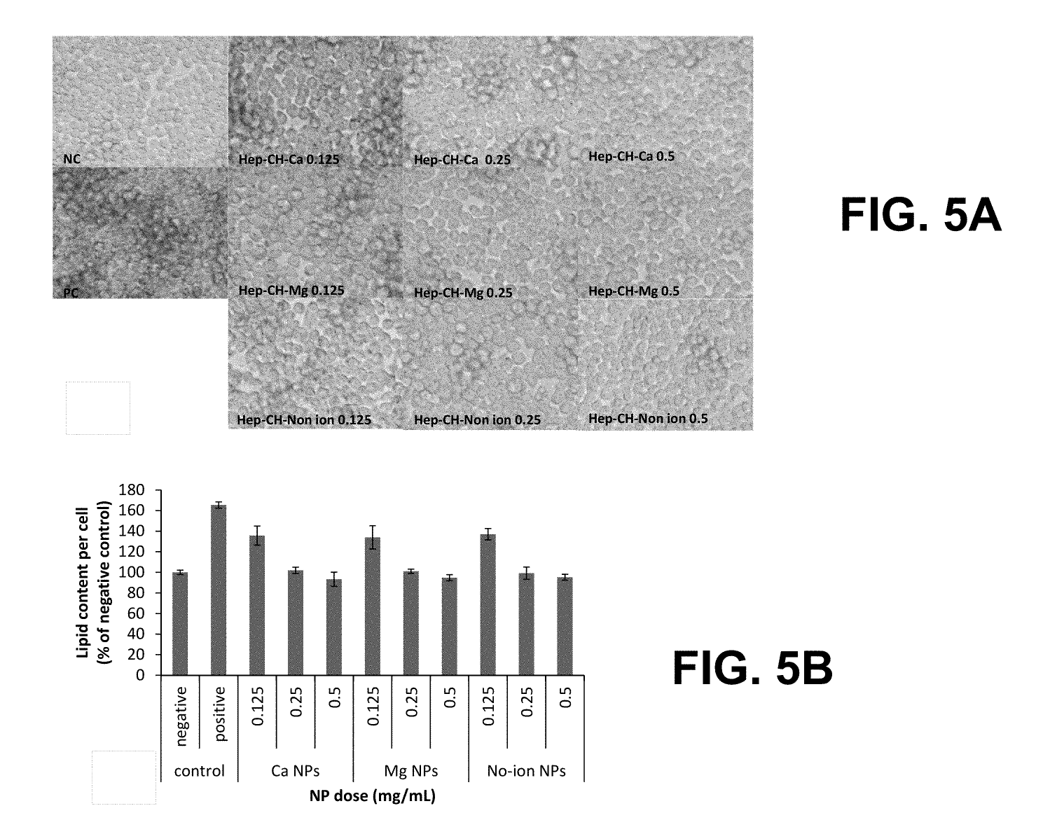

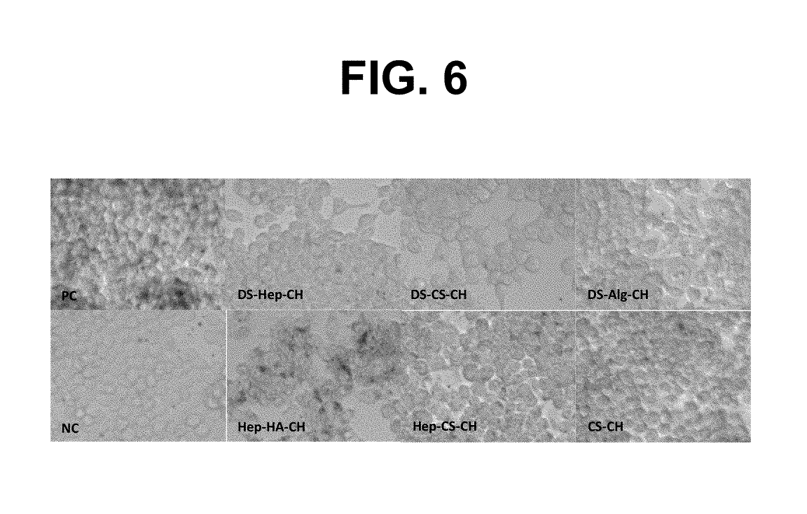

To examine whether nanoparticles could reduce foam cell formation by binding to oxLDL (Intracel), macrophage cell culture medium containing 20 μg/mL oxLDL was incubated with nanoparticles for 24 h at 37° C. and centrifuged at 10,000 rpm for 10 min. The supernatant was filtered through a 0.22 μm filter and remove any remaining nanoparticles and was used to treat macrophages for 24 h. Cells treated with culture medium in the presence or absence of oxLDL without NP treatment were used as positive and negative controls, respectively. The culture medium for the control cultures was also incubated at 37° C. and filtered before it was used for cell culture. Foam cell formation was assessed by Oil red O staining and quantification of the cellular cholesteryl ester (CE) content or lipid content. See, FIGS. 3-6. Nanoparticle could effectively remove oxLDL from the culture medium.

### Example 6

### Inhibition of OxLDL Internalization by Macrophages (Co-Treatment and Pre-Treatment)

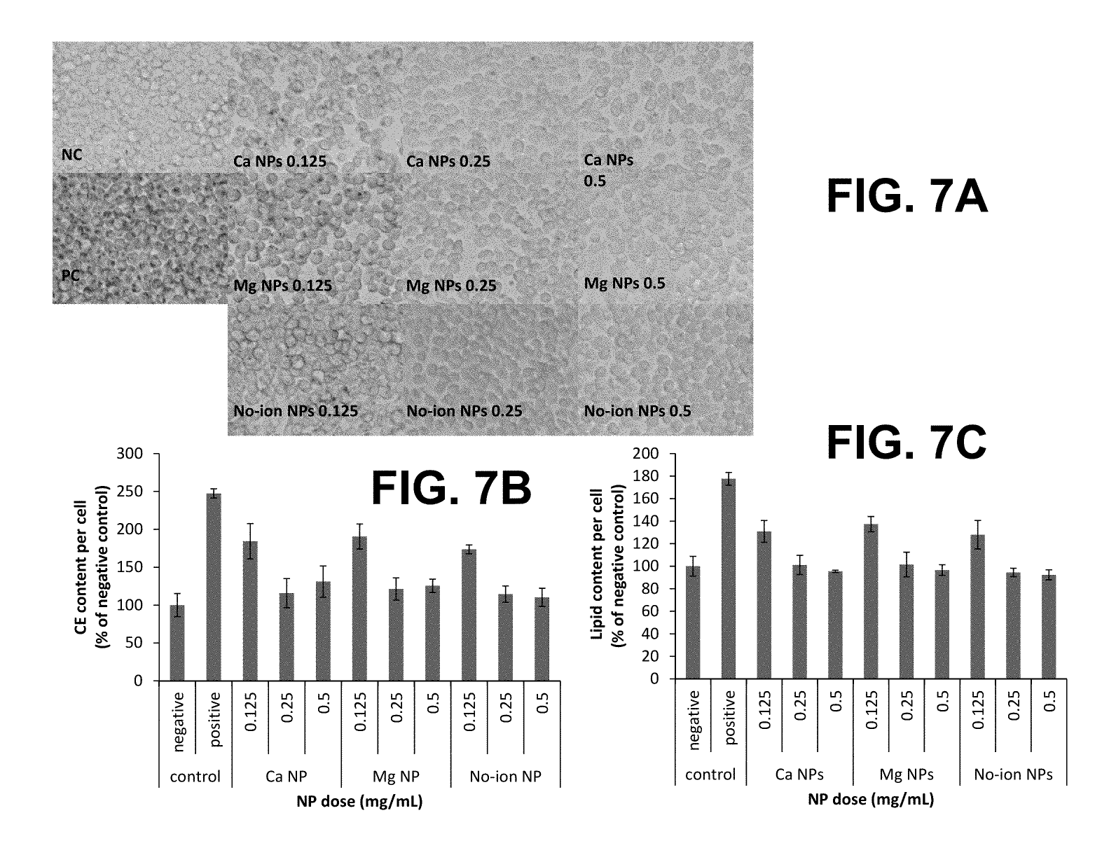

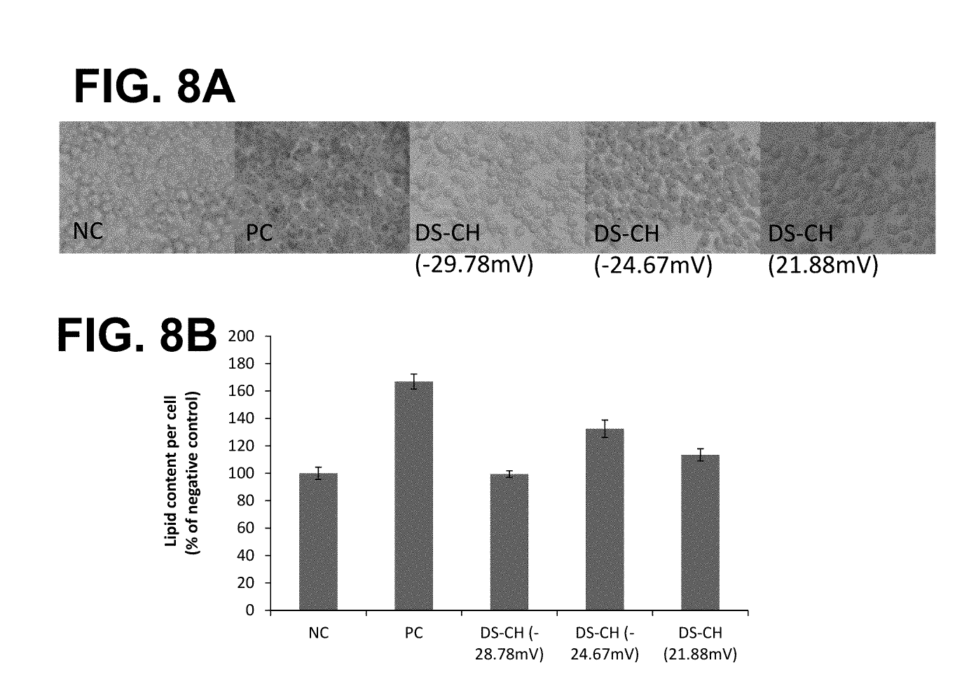

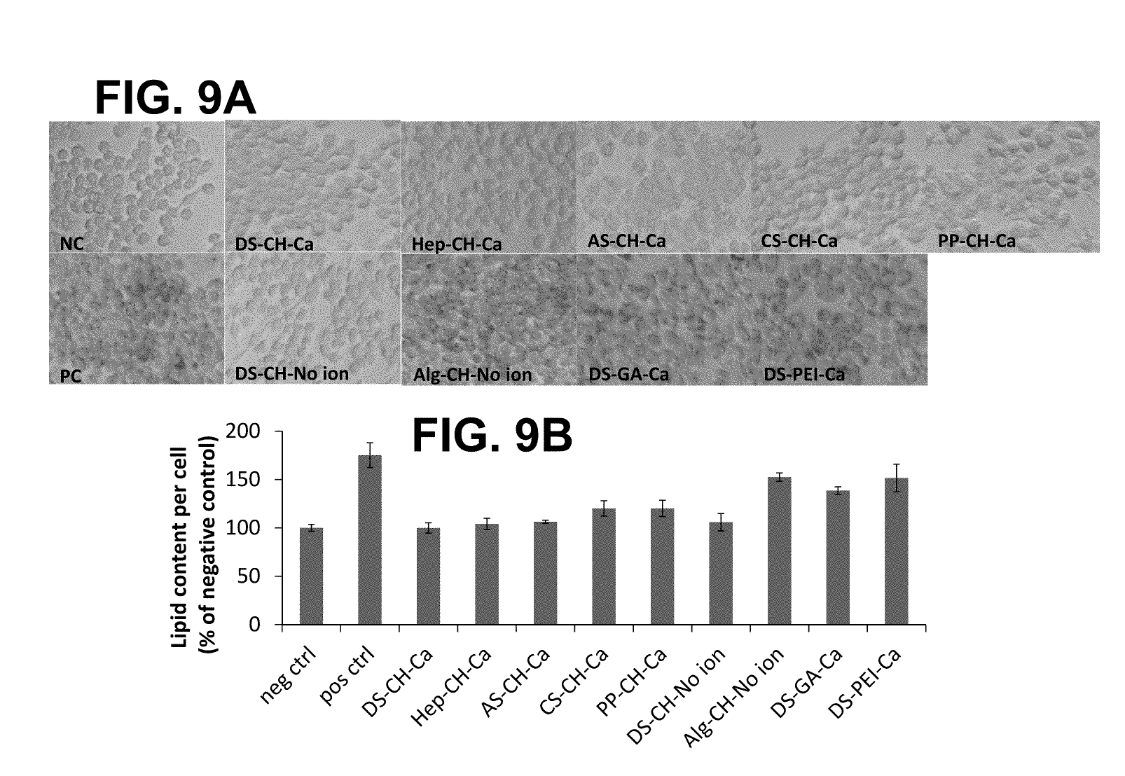

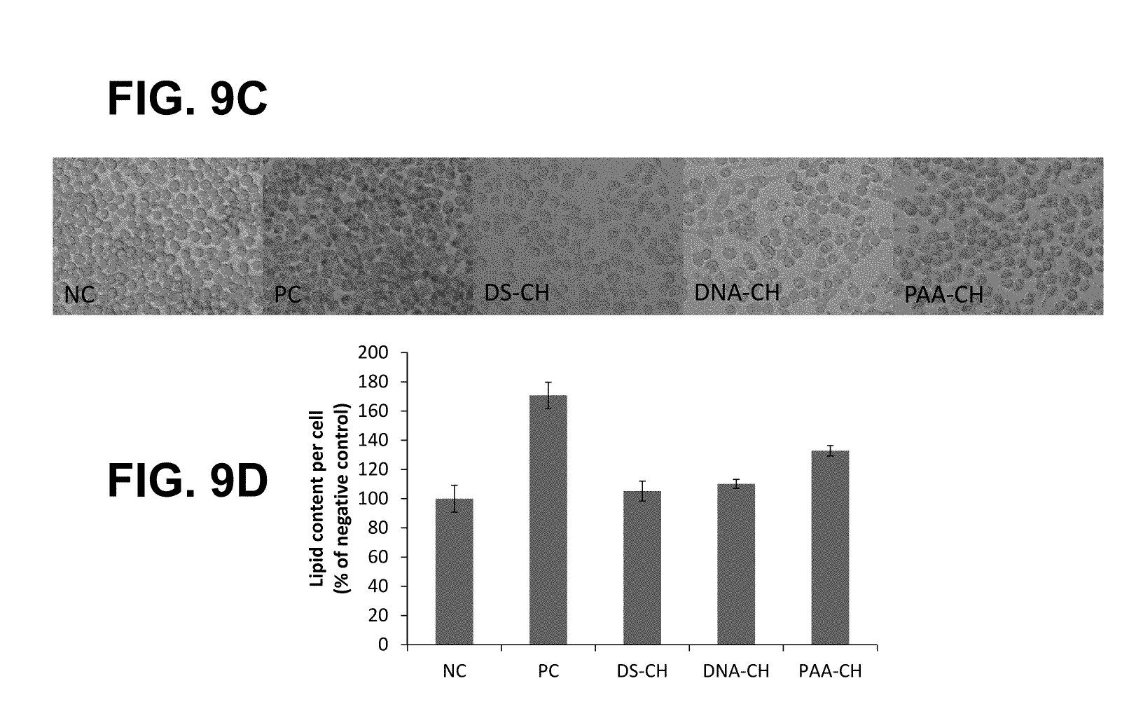

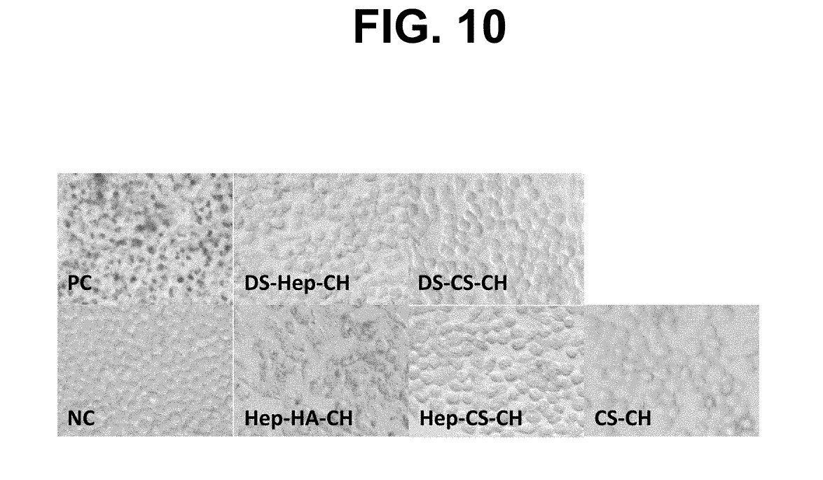

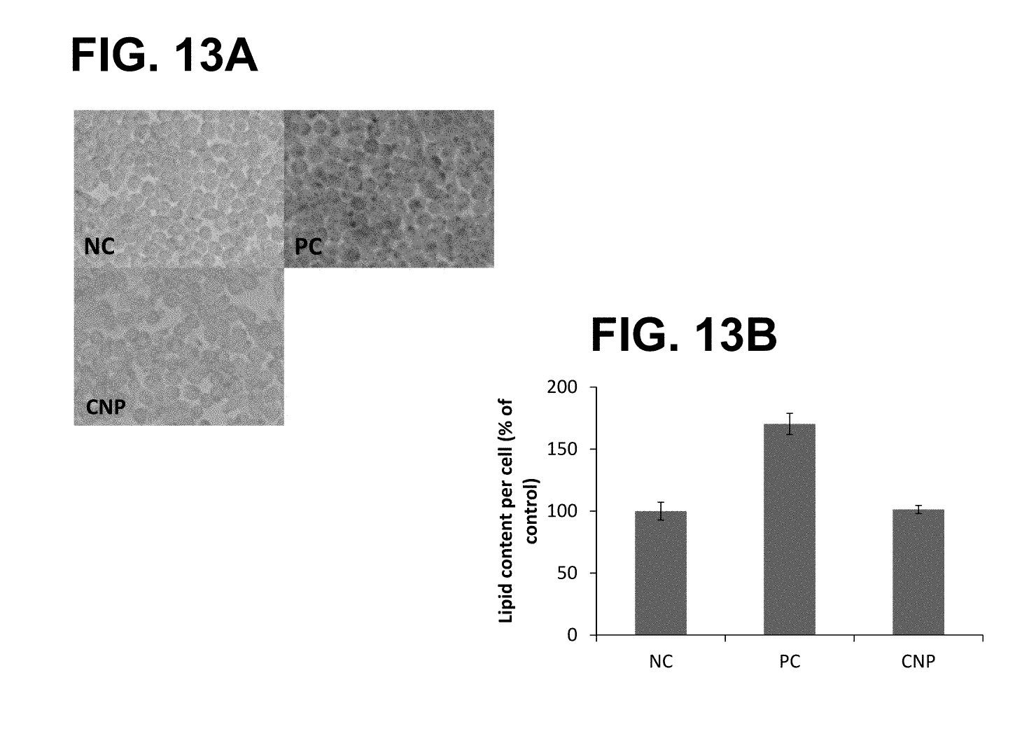

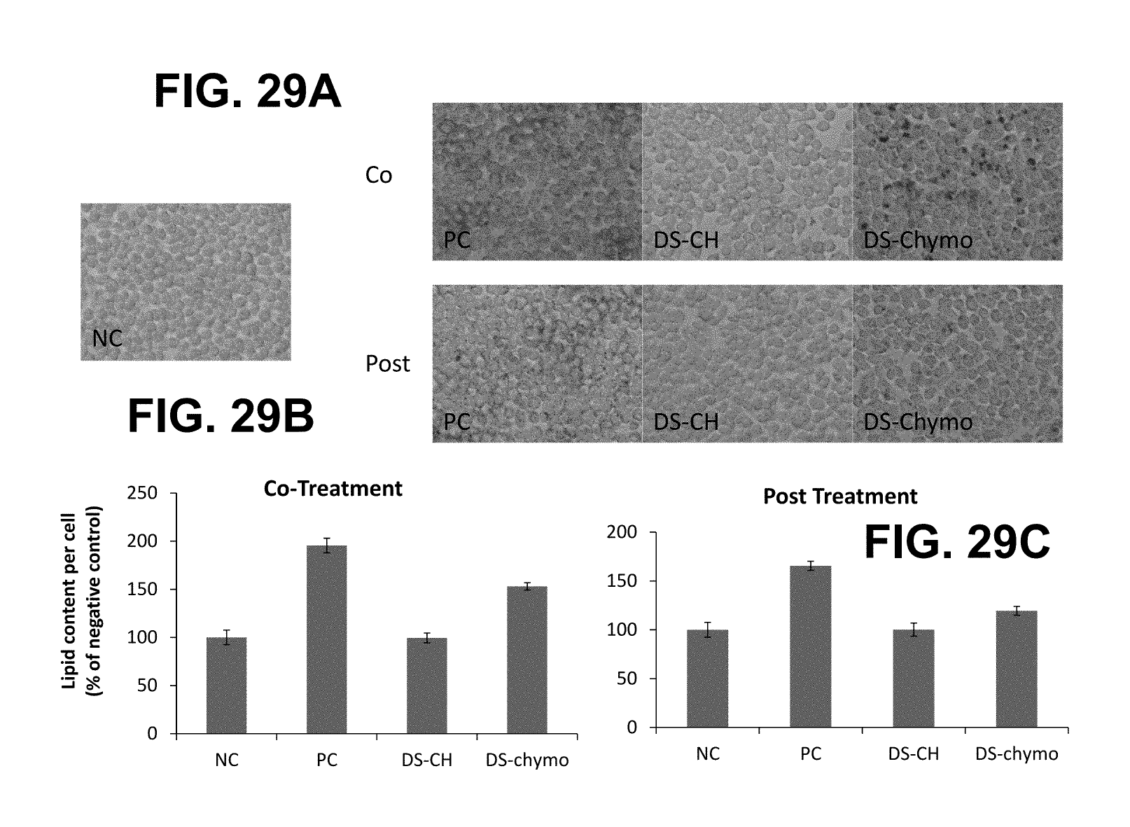

Macrophages were incubated with oxLDL and nanoparticles at the same time (co-treatment) to study whether the presence of nanoparticles can inhibit oxLDL uptake. Because in the body the nanoparticles will eventually be removed from the blood stream, NP treatment was studied to determine if it had any long lasting effect after the nanoparticles are removed (pre-treatment). Mouse RAW264.7 macrophages were cultured as described above. Human monocyte THP-1 cells were cultured in RPMI 1640 medium supplemented with 10% FBS and 1% P/S. The monocytes were differentiated into macrophages by incubating with 320 nM PMA overnight prior to treatment. All the cells were maintained in a 37° C., 5% CO₂ incubator. For co-treatment, mouse macrophages were incubated with 30 (Intracel) or 50 (Alfa Aesar) μg/mL oxLDL and nanoparticles simultaneously for 24 h. See, FIGS. 7-10, 13, 29. Nanoparticles could effectively inhibit oxLDL uptake by macrophages.

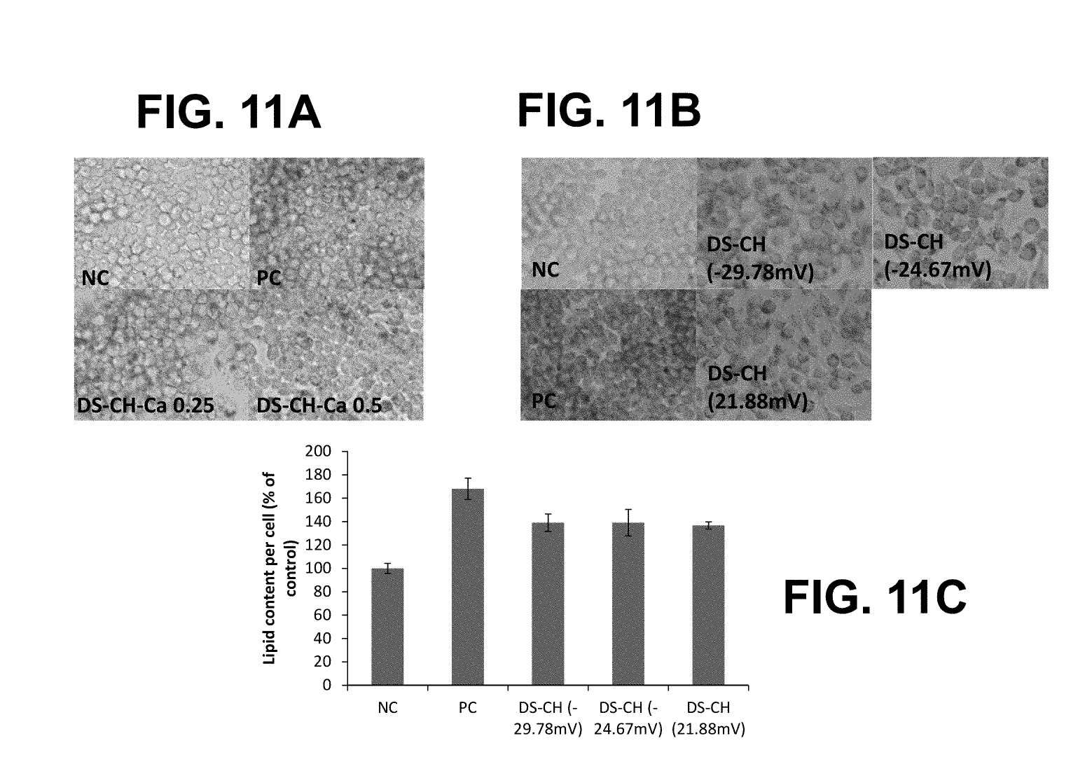

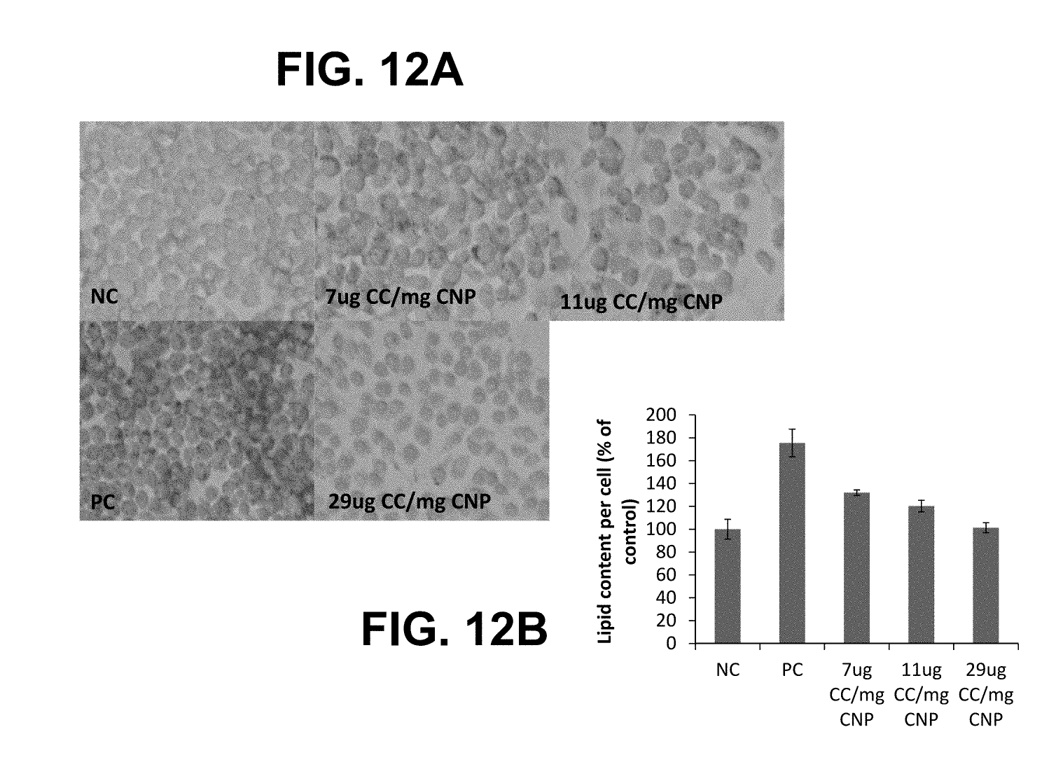

For pre-treatment mouse RAW264.7 macrophages were incubated with the nanoparticles for 24 h prior to oxLDL or spinal cord homogenate treatment. After 24 h of NP treatment, the cells were thoroughly washed to remove any nanoparticles in the culture medium, and then the cells were treated with 25 μg/mL oxLDL (Alfa Aesar) or spinal cord homogenate for 24 h. Positive and negative control groups were treated with and without oxLDL without NP treatment. See, FIGS. 11, 12, and 26. Nanoparticles could effectively inhibit oxLDL uptake by macrophages after the nanoparticles were removed from the culture medium. It is noteworthy that the curcumin used in FIG. 26 was Sigma 1386.

### Example 7

### Promotion of Cholesterol Efflux (Post-Treatment)

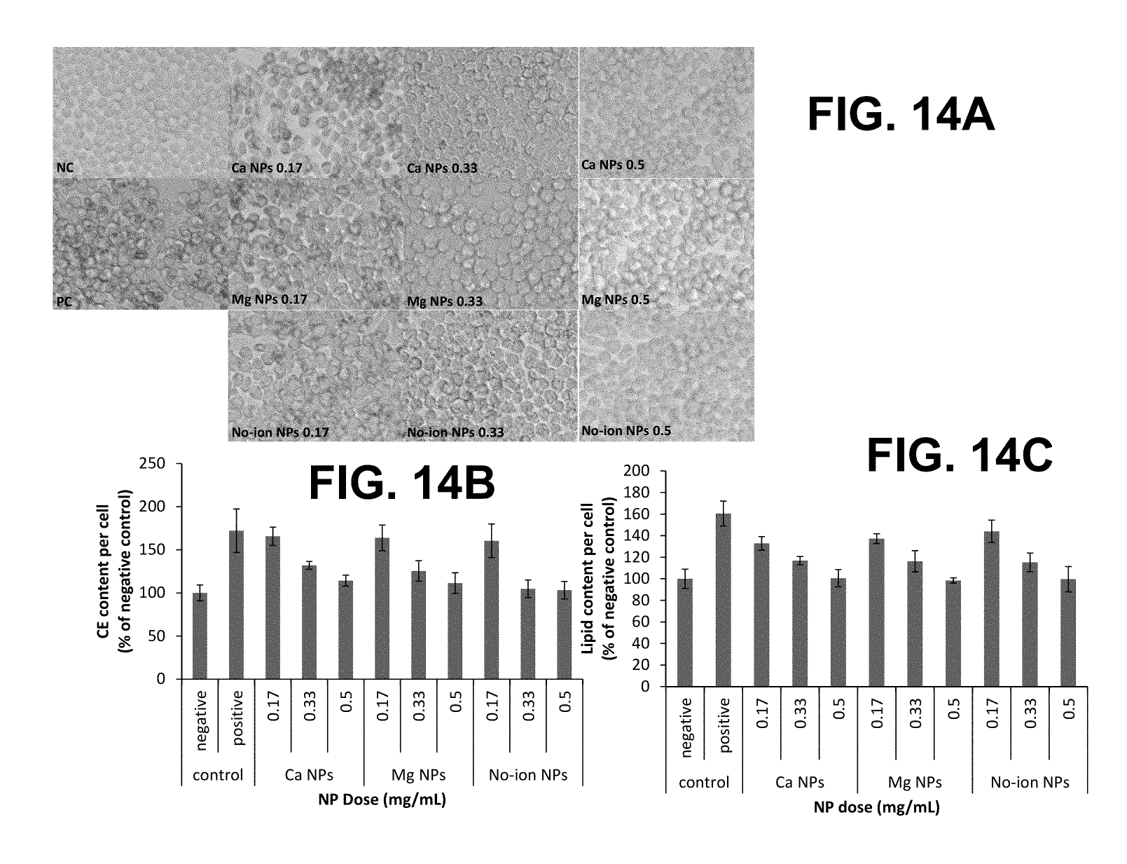

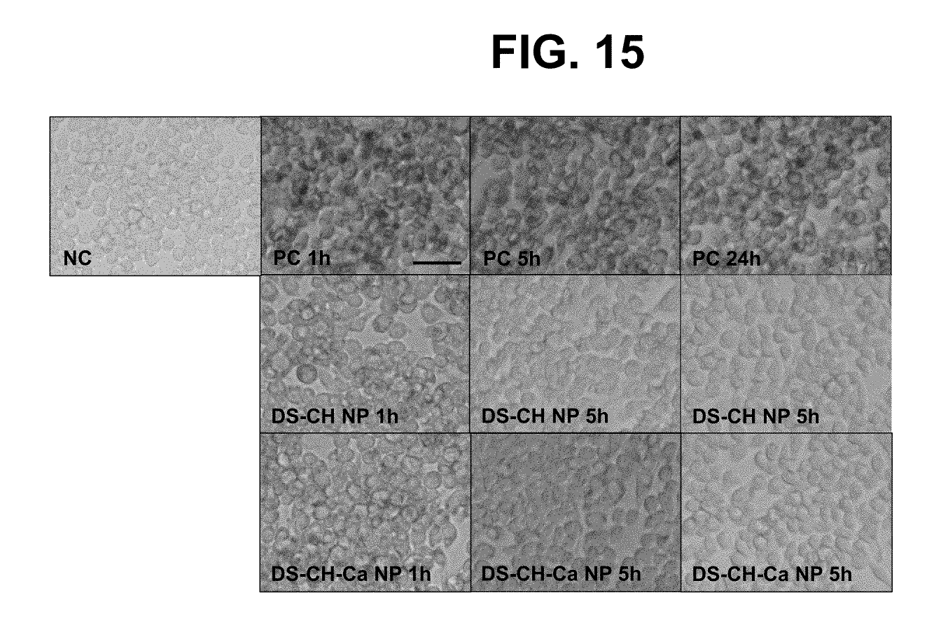

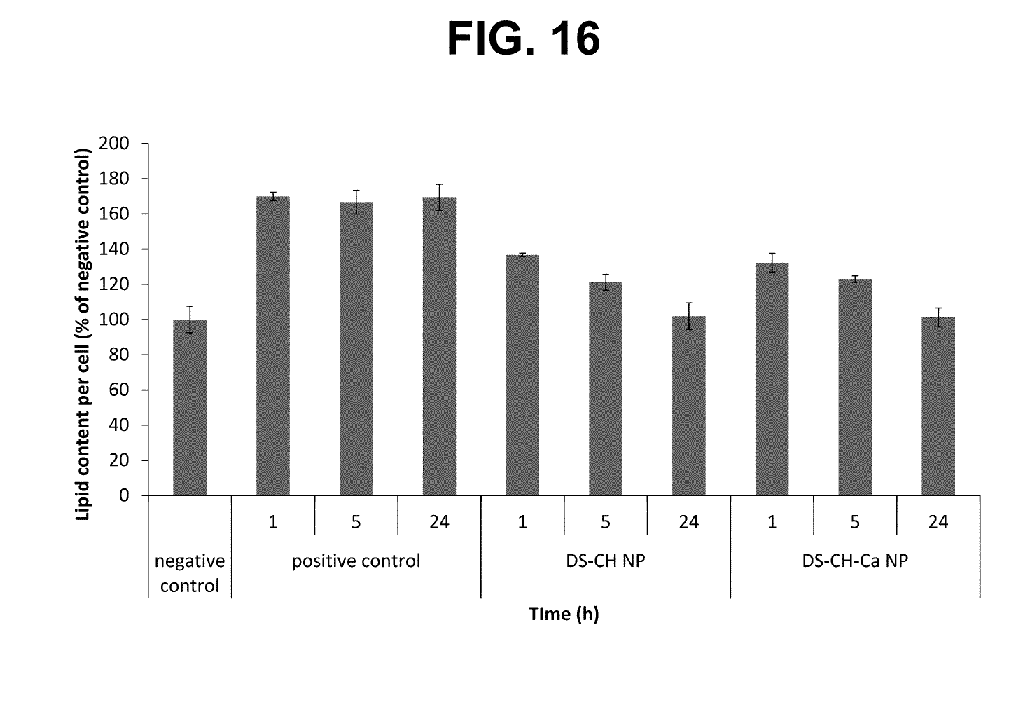

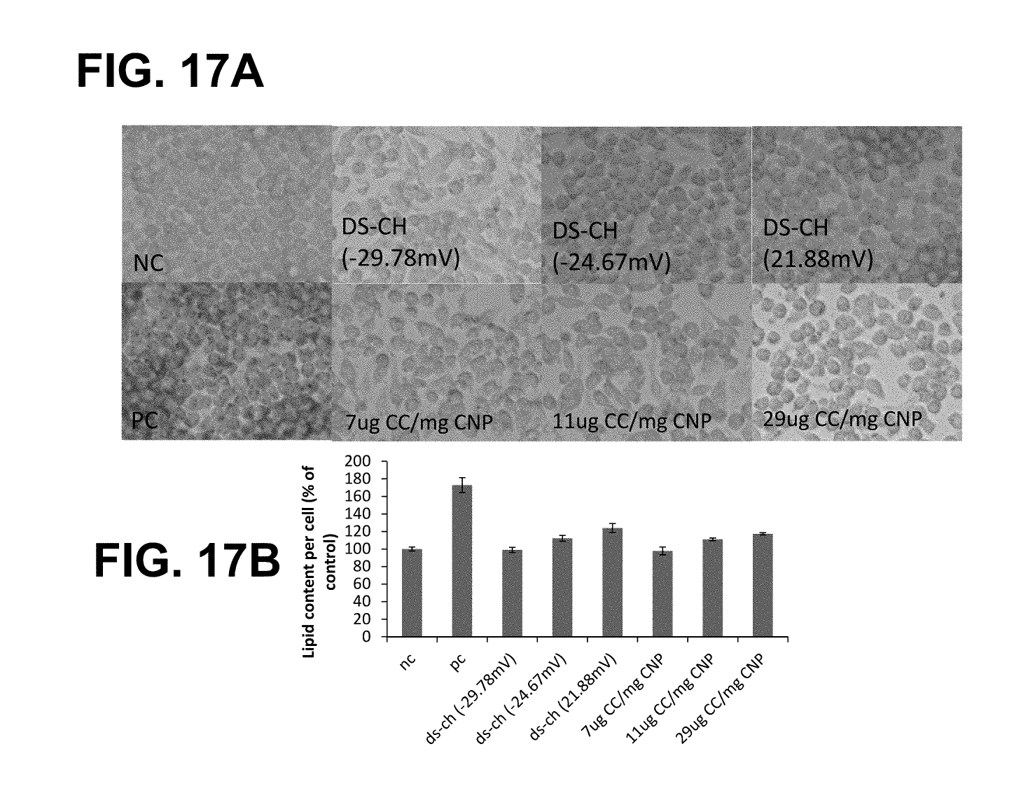

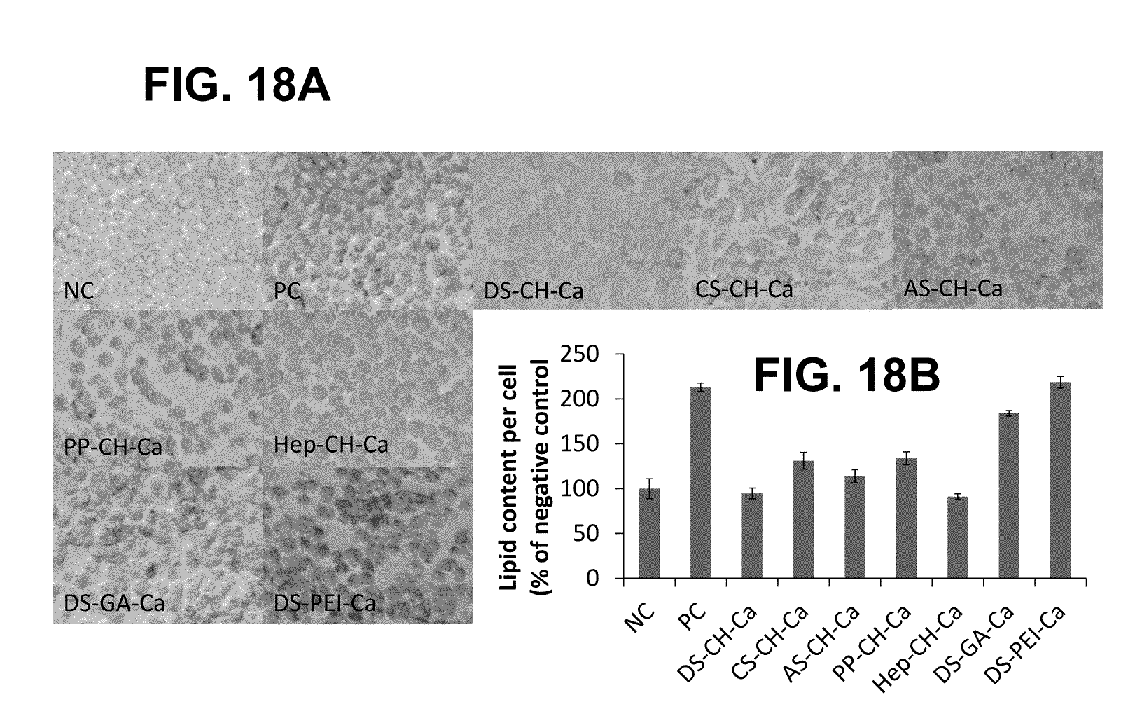

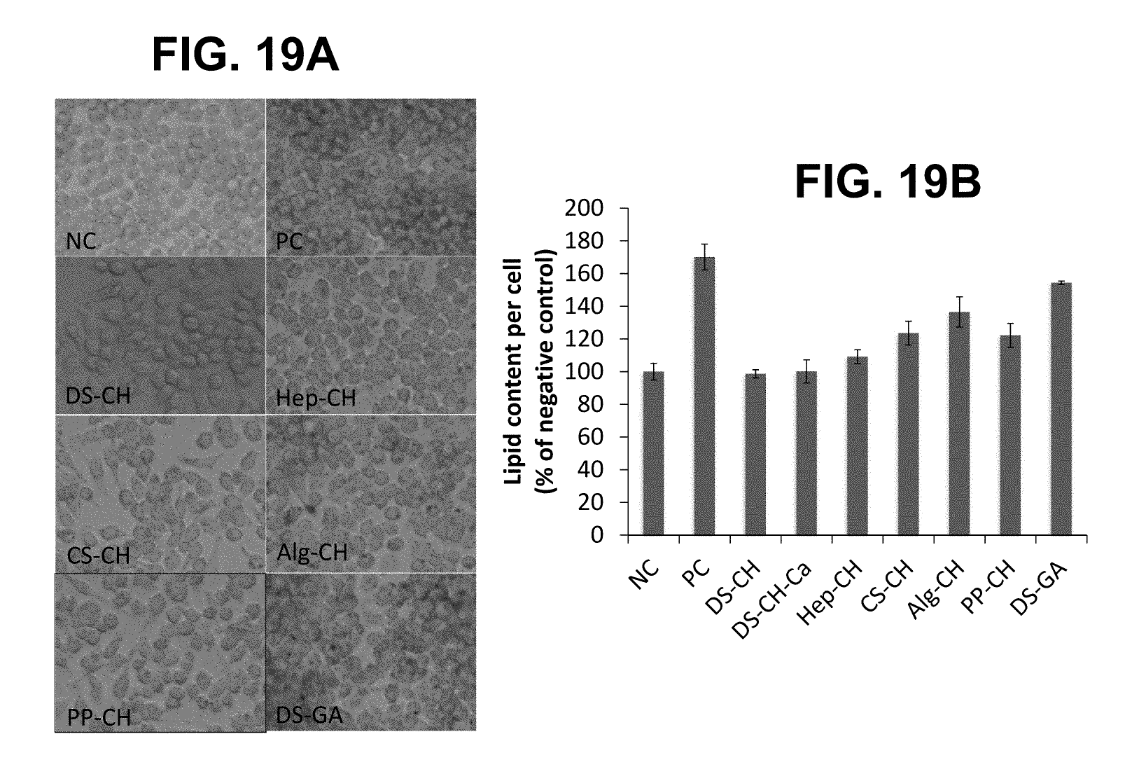

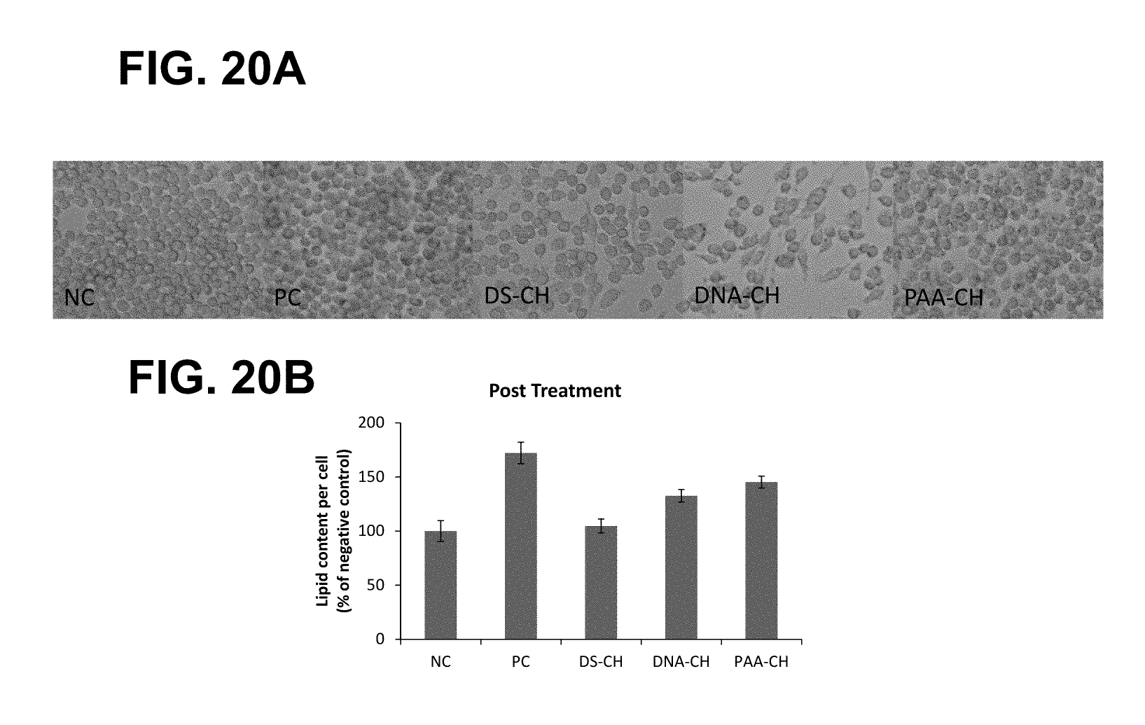

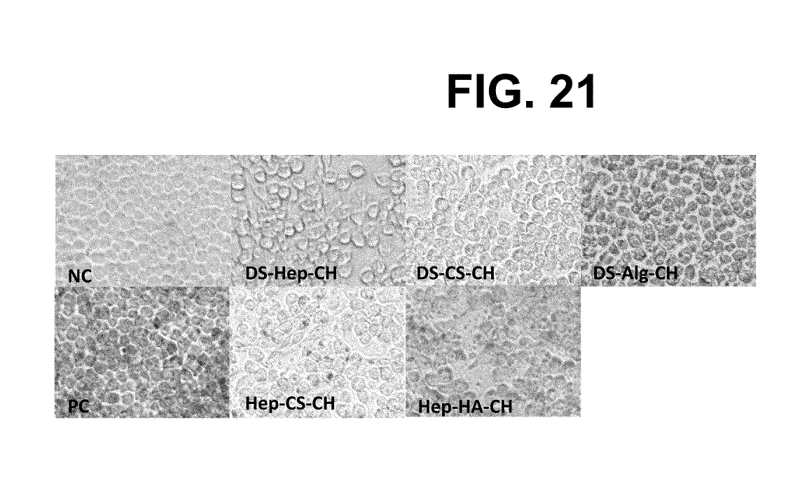

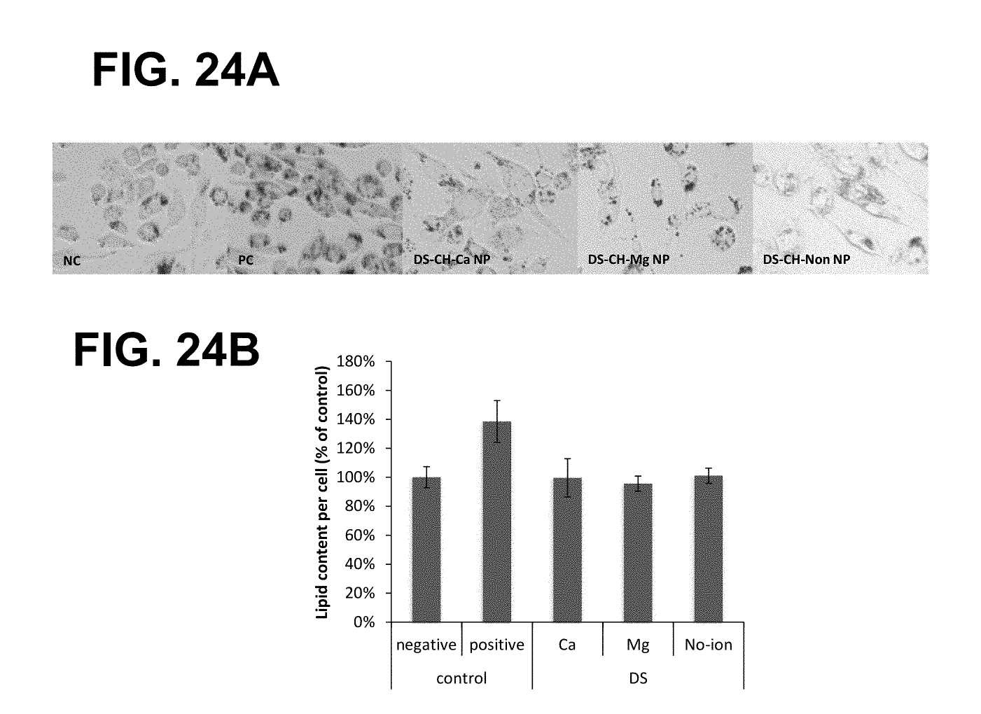

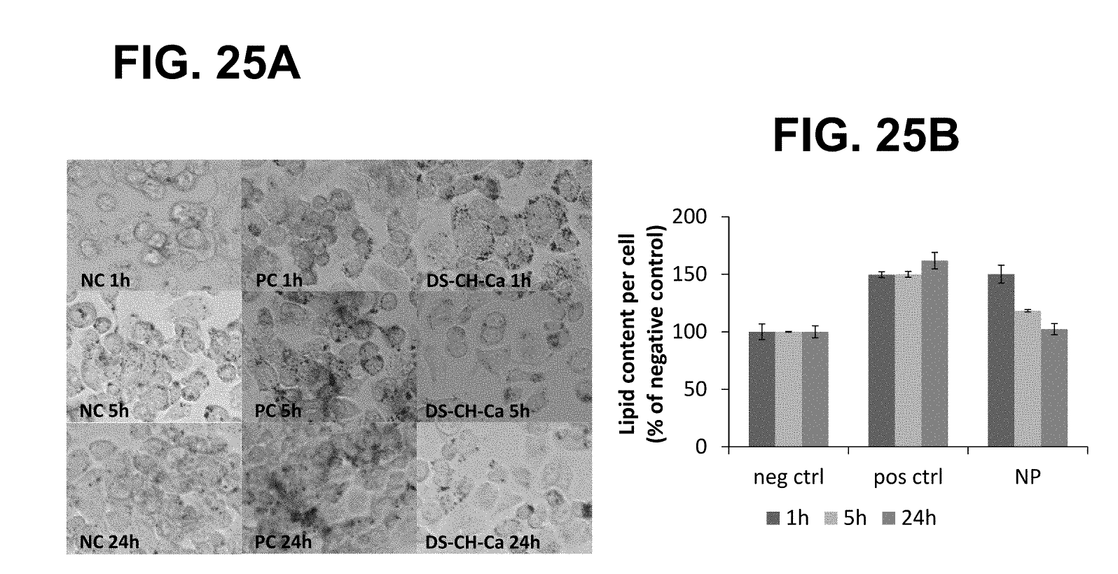

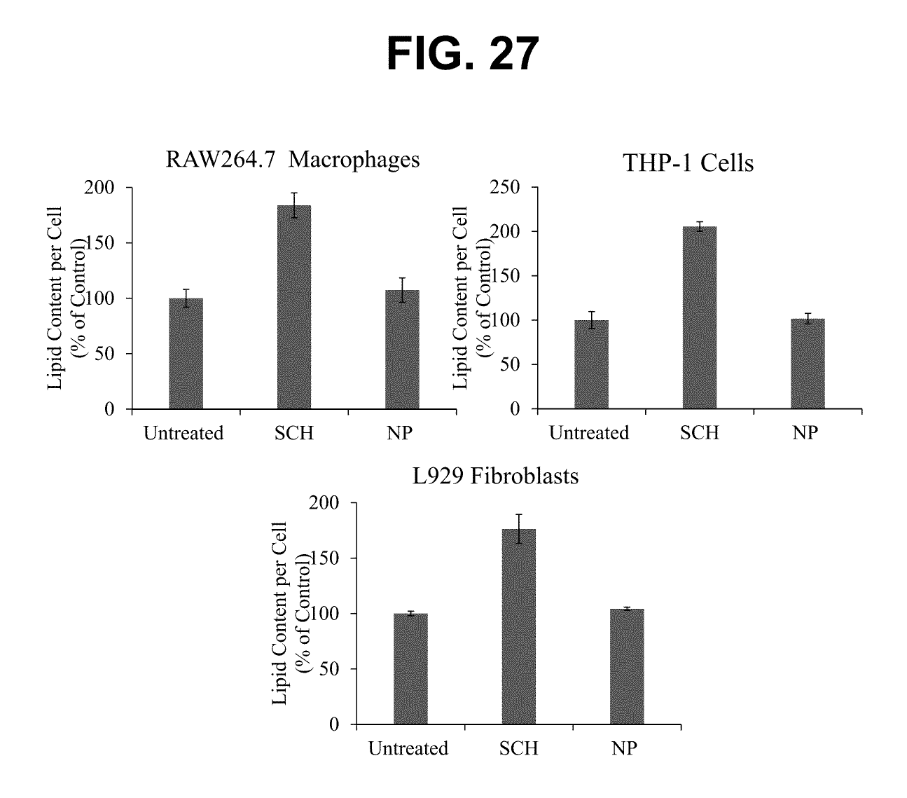

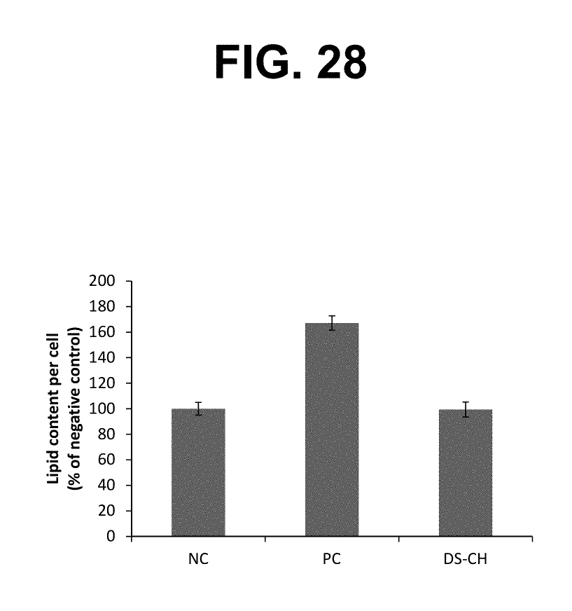

Mouse macrophages were treated with 20 (Intracel) or 25 μg/mL (Alfa Aesar) oxLDL, or spinal cord homogenate, or mylin debris for 24 h to induce foam cell formation. Human macrophages were treated with 30 μg/mL oxLDL for 24 h to induce foam cell formation. Foam cells were then treated with nanoparticles to induce cholesterol efflux. Foam cells incubated in culture medium without nanoparticles were used as positive control. Cells without any treatment were used as negative control. See FIGS. 14-21, 24, 25, and 27-29. Most nanoparticles could effectively induce cholesterol efflux from foam cells.

### Example 8

### Oil Red Staining, Quantification of Lipid Content and Cholesteryl Ester (CE) Level of Foam Cells

Macrophages were washed twice with PBS and fixed in 4% paraformaldehyde for 15 min. After washing twice with PBS, the fixed cells were treated with 60% isopropanol for 5 min, followed by treating with 2.4% Oil Red O solution for 15 min. The cells were then washed with 60% isopropanol for 5 sec and kept in PBS for imaging. After imaging the cells were treated with 100% isopropanol for 1 min to extract oil red O, which was measured photometrically at 520 nm using a Tecan M200 microplate reader to assess lipid content. The cellular cholesteryl ester (CE) level was quantified using an Amplex red Cholesterol Assay kit (Invitrogen) following the manufacturer's instructions.

### Example 9

### Cholesterol Binding Assay

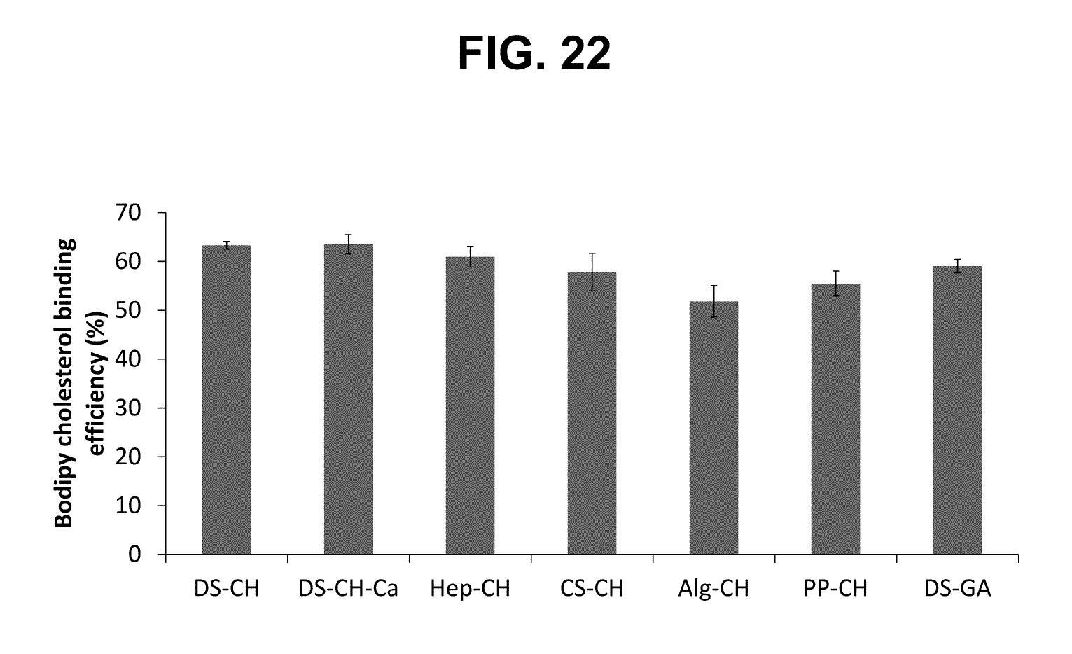

The ability of the nanoparticles to bind to cholesterol was assessed by mixing the nanoparticles in 100 μL of DI water with 0.25 μL of 2 mg/mL BODIPY cholesterol (Avanti), solution followed by incubation for 4 h. Then the mixture was centrifuged at 3,000 rpm for 3 minutes, the unbound cholesterol level in the supernatant was measured at excitation of 485 nm and emission at 535 nm for the calculation of cholesterol binding efficiency. See, FIG. 22. Nanoparticles could effectively bind to cholesterol.

### Example 10

### Anti-Inflammatory Activity Assay

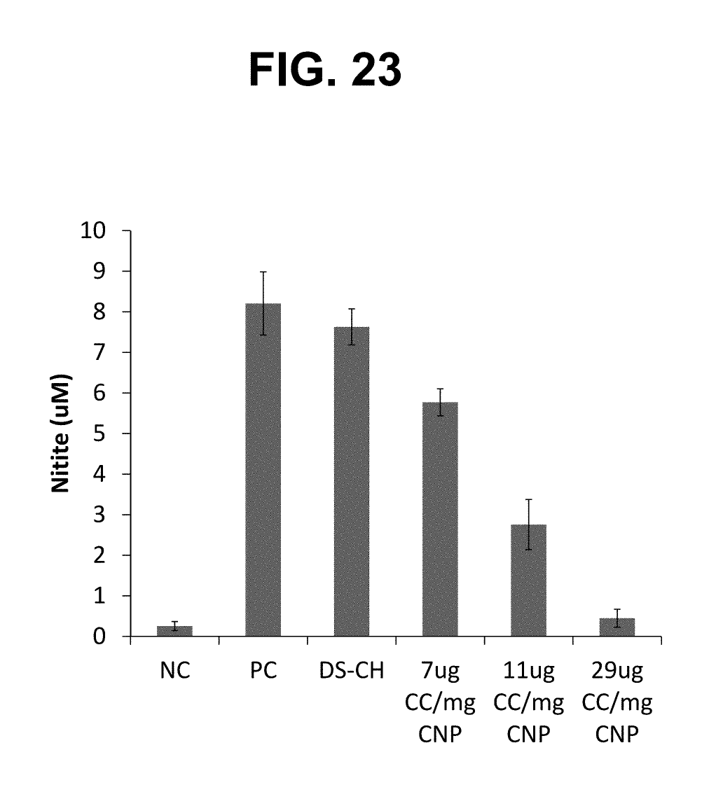

RAW 264.7 mouse macrophages were treated with 200 pg/mL LPS and with 0.5 mg/mL NP. After 48 h, the accumulated levels of nitrite in the cell culture medium, as an indication of NO, were measured using Griess reagent (Sigma). Cell treated with medium in the presence or absence of LPS without other treatments were considered as positive control (PC) and negative control (NC), respectively. See, FIG. 23. DS-CH nanoparticles did not have any significant effect on LPS-induced NO production, whereas CNP significantly reduced NO production.

It is to be understood that while the invention has been described in conjunction with the preferred specific embodiments thereof, that the foregoing description and the examples that follow are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains. In addition to the embodiments described herein, the present invention contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

The disclosures of each patent, patent application, and publication cited or described in this document are hereby incorporated herein by reference, each in its entirety, for all purposes.

What is claimed:

1. Nanoparticles having a hydrodynamic diameter of from about 10 to about 400 nm, comprising a polymer comprising positively charged polymer and at least one anionic polymer comprising sulfate groups, phosphate groups, carboxyl groups, or a combination thereof.

2. The nanoparticles of claim 1, wherein said at least one anionic polymer comprises sulfate groups.

3. The nanoparticles of claim 2, wherein said at least one anionic polymer is dextran sulfate, cellulose sulfate, heparin, heparin sulfate, chondroitin sulfate, dermatan sulfate, keratan sulfate, alginate sulfate, aggrecan, fucoidan, or polystyrene sulfonate.

4. The nanoparticles of claim 1, wherein said at least one anionic polymer comprises phosphate groups.

5. The nanoparticles of claim 4, wherein said at least one anionic polymer comprises polyphosphate, DNA, or RNA.

6. The nanoparticles of claim 1, wherein said at least one anionic polymer comprises carboxyl groups.

7. The nanoparticles of claim 6, wherein said at least one anionic polymer is hyaluronic acid, pectin, carboxymethyl dextran, carboxymethyl amylose, carboxymethyl cellulose, carboxymethyl beta-cyclodextrin, poly(acrylic acid), or combinations thereof.

8. The nanoparticles of claim 1, wherein polymer comprising positively charged groups is chitosan, glycol chitosan, gelatin type A, or PEI.

9. The nanoparticles of claim 8, wherein the chitosan has an average molecular weight (M_{w}) of from about 10 to about 400 kDa.

10. The nanoparticles of claim 1, wherein the at least one anionic polymer has a M_{w} of greater than about 1 kDa.

11. The nanoparticles of claim 1, further comprising an active agent that is an antibiotic, an oxygen scavenger, anti-inflammatory, low-density lipoprotein (LDL) anti-oxidant, agent that reduces uptake of oxidized LDL, agent that increases high-density lipoprotein (HDL) release, or combinations thereof.

12. The nanoparticles of claim 11, wherein the active agent is minocycline or curcumin.

13. The nanoparticles of claim 1, comprising:
(i) dextran sulfate, heparin, alginate sulfate, alginate, chondroitin sulfate, polyphosphate, and chitosan;
(ii) dextran sulfate, chondroitin sulfate and chitosan;
(iii) dextran sulfate, alginate, and chitosan;
(iv) chondroitin sulfate, hyaluronic acid, and heparin;
(v) heparin, chondroitin sulfate, and chitosan; or
(vi) heparin, hyaluronic acid, and chitosan.

14. The nanoparticles of claim 1, further comprising a metal ion.

15. The nanoparticles of claim 14, wherein said metal ion is a monovalent or divalent metal ion.

16. The nanoparticles of claim 15, wherein said monovalent ion is NaCl.

17. The nanoparticles of claim 15, wherein said divalent metal ion is an alkaline earth metal.

18. The nanoparticles of claim 17, wherein said alkaline earth metal is Ca²⁺ or Mg²⁺.

19. A pharmaceutical composition comprising the nanoparticles of claim 1 and a pharmaceutically acceptable excipient.

20. A method for preparing nanoparticles having a hydrodynamic diameter of from about 10 to about 400 nm, comprising a chitosan, gelatin type A, or PEI and at least one anionic polymer, said method comprising
(i) mixing said chitosan with an acid to form a solution;
(ii) adding the solution of step (i) to a solution comprising said at least one anionic polymer;
(iii) stirring the product of step (ii); and
(iv) isolating said nanoparticles.

21. A method for treating atherosclerosis, or preventing the progression of atherosclerosis, inhibiting oxidized LDL uptake by macrophages, reducing low density lipoprotein levels, elevating apolipoprotein-A1 (ApoA1) production by foam cells, or preventing foam cell formation in a subject, comprising administering said nanoparticles of claim 1 to said subject.

22. A method of inducing cholesterol efflux from foam cells or removing effluxed cholesterol from cells in a subject, comprising administering nanoparticles to said subject, the nanoparticles having a hydrodynamic diameter of from about 10 to about 400 nm, comprising a positively charged polymer comprising a hydrophobic component and at least one anionic polymer comprising sulfate groups, phosphate groups, carboxyl groups, or a combination thereof.

23. The method of claim 22, wherein the nanoparticles bind to the cholesterol.

24. A method of treating spinal cord injury in a subject, comprising administering the nanoparticles of claim 1 to said subject.
